(19) United States
(12) Patent Application Publication
(10) Pub. No.: US 2022/0009945 A1
(43) Pub. Date: Jan. 13, 2022

(54) NOVEL UREA 6,7-DIHYDRO-4H-THIAZOLE[5,4-C]PYRIDINES ACTIVE AGAINST THE HEPATITIS B VIRUS (HBV)

(71) Applicant: AICURIS GMBH & CO. KG, Wuppertal (DE)

(72) Inventors: Alastair DONALD, Wuppertal (DE); Andreas URBAN, Sprockhövel (DE); Susanne BONSMANN, Köln (DE)

(73) Assignee: AICURIS GMBH & CO. KG, Wuppertal (DE)

(21) Appl. No.: 17/290,504

(22) PCT Filed: Nov. 1, 2019
(86) PCT No.: PCT/EP2019/079982

§ 371 Date: Apr. 30, 2021

(30) Foreign Application Priority Data
Nov. 2, 2018 (EP) 18000875.7

(51) Int. Cl. C07D 513/04

(52) U.S. Cl. CPC C07D 513/04

(57) ABSTRACT

The present invention relates generally to novel antiviral agents. Specifically, the present invention relates to compounds which can inhibit the protein(s) encoded by hepatitis B virus (HBV) or interfere with the function of the HBV replication cycle, compositions comprising such compounds, methods for inhibiting HBV viral replication, methods for treating or preventing HBV infection, and processes and intermediates for making the compounds.

### TECHNICAL FIELD

The present invention relates generally to novel antiviral agents. Specifically, the present invention relates to compounds which can inhibit the protein(s) encoded by hepatitis B virus (HBV) or interfere with the function of the HBV replication cycle, compositions comprising such compounds, methods for inhibiting HBV viral replication, methods for treating or preventing HBV infection, and processes for making the compounds.

### BACKGROUND OF THE INVENTION

Chronic HBV infection is a significant global health problem, affecting over 5% of the world population (over 350 million people worldwide and 1.25 million individuals in the US). Despite the availability of a prophylactic HBV vaccine, the burden of chronic HBV infection continues to be a significant unmet worldwide medical problem, due to suboptimal treatment options and sustained rates of new infections in most parts of the developing world. Current treatments do not provide a cure and are limited to only two classes of agents (interferon alpha and nucleoside analogues/inhibitors of the viral polymerase); drug resistance, low efficacy, and tolerability issues limit their impact.

The low cure rates of HBV are attributed at least in part to the fact that complete suppression of virus production is difficult to achieve with a single antiviral agent, and to the presence and persistence of covalently closed circular DNA (cccDNA) in the nucleus of infected hepatocytes. However, persistent suppression of HBV DNA slows liver disease progression and helps to prevent hepatocellular carcinoma (HCC).

Current therapy goals for HBV-infected patients are directed to reducing serum HBV DNA to low or undetectable levels, and to ultimately reducing or preventing the development of cirrhosis and HCC.

The HBV is an enveloped, partially double-stranded DNA (dsDNA) virus of the hepadnavirus family (Hepadnaviridae). HBV capsid protein (HBV-CP) plays essential roles in HBV replication. The predominant biological function of HBV-CP is to act as a structural protein to encapsidate pre-genomic RNA and form immature capsid particles, which spontaneously self-assemble from many copies of capsid protein dimers in the cytoplasm.

HBV-CP also regulates viral DNA synthesis through differential phosphorylation states of its C-terminal phosphorylation sites. Also, HBV-CP might facilitate the nuclear translocation of viral relaxed circular genome by means of the nuclear localization signals located in the arginine-rich domain of the C-terminal region of HBV-CP.

In the nucleus, as a component of the viral cccDNA mini-chromosome, HBV-CP could play a structural and regulatory role in the functionality of cccDNA mini-chromosomes. HBV-CP also interacts with viral large envelope protein in the endoplasmic reticulum (ER), and triggers the release of intact viral particles from hepatocytes.

HBV-CP related anti-HBV compounds have been reported. For example, phenylpropenamide derivatives, including compounds named AT-61 and AT-130 (Feld J. et al. Antiviral Res. 2007, 76, 168), and a class of thiazolidin-4-ones from Valeant (WO2006/033995), have been shown to inhibit pre-genomic RNA (pgRNA) packaging.

F. Hoffmann-LA Roche AG have disclosed a series of 3-substituted tetrahydro-pyrazolo[1,5-a]pyrazines for the therapy of HBV (WO2016/113273, WO2017/198744, WO2018/011162, WO2018/011160, WO2018/011163).

Heteroaryldihydropyrimidines (HAPs) were discovered in a tissue culture-based screening (Weber et al., Antiviral Res. 2002, 54, 69). These HAP analogs act as synthetic allosteric activators and are able to induce aberrant capsid formation that leads to degradation of HBV-CP (WO 99/54326, WO 00/58302, WO 01/45712, WO 01/6840). Further HAP analogs have also been described (J. Med. Chem. 2016, 59 (16), 7651-7666).

A subclass of HAPs from F. Hoffman-La Roche also shows activity against HBV (WO2014/184328, WO2015/132276, and WO2016/146598). A similar subclass from Sunshine Lake Pharma also shows activity against HBV (WO2015/144093). Further HAPs have also been shown to possess activity against HBV (WO2013/102655, Bioorg. Med. Chem. 2017, 25(3) pp. 1042-1056, and a similar subclass from Enanta Therapeutics shows similar activity (WO2017/011552). A further subclass from Medshine Discovery shows similar activity (WO2017/076286). A further subclass (Janssen Pharma) shows similar activity (WO2013/102655).

A subclass of pyridazones and triazinones (F. Hoffman-La Roche) also show activity against HBV (WO2016/023877), as do a subclass of tetrahydropyridopyridines (WO2016/177655). A subclass of tricyclic 4-pyridone-3-carboxylic acid derivatives from Roche also show similar anti-HBV activity (WO2017/013046).

A subclass of sulfamoyl-arylamides from Novira Therapeutics (now part of Johnson & Johnson Inc.) also shows activity against HBV (WO2013/006394, WO2013/096744, WO2014/165128, WO2014/184365, WO2015/109130, WO2016/089990, WO2016/109684, WO2016/109689, WO2017/059059).

A similar subclass of thioether-arylamides (also from Novira Therapeutics) shows activity against HBV (WO2016/089990). Additionally, a subclass of aryl-azepanes (also from Novira Therapeutics) shows activity against HBV (WO2015/073774). A similar subclass of arylamides from Enanta Therapeutics show activity against HBV (WO2017/015451).

Sulfamoyl derivatives from Janssen Pharma have also been shown to possess activity against HBV (WO2014/033167, WO2014/033170, WO2017001655, J. Med. Chem, 2018, 61(14) 6247-6260)

A subclass of glyoxamide substituted pyrrolamide derivatives also from Janssen Pharma have also been shown to possess activity against HBV (WO2015/011281). A similar class of glyoxamides from Gilead Sciences also possess activity against HBV (WO2018/039531).

A subclass of sulfamoyl- and oxalyl-heterobiaryls from Enanta Therapeutics also show activity against HBV (WO2016/161268, WO2016/183266, WO2017/015451, WO2017/136403 & US20170253609).

A subclass of aniline-pyrimidines from Assembly Biosciences also show activity against HBV (WO2015/057945, WO2015/172128). A subclass of fused tri-cycles from Assembly Biosciences (dibenzo-thiazepinones, dibenzo-diazepinones, dibenzo-oxazepinones) show activity against HBV (WO2015/138895, WO2017/048950).

A series of cyclic sulfamides has been described as modulators of HBV-CP function by Assembly Biosciences (WO2018/160878).

Arbutus Biopharma have disclosed a series of benzamides for the therapy of HBV (WO2018/052967, WO2018/172852).

It was also shown that the small molecule bis-ANS acts as a molecular ‘wedge’ and interferes with normal capsid-protein geometry and capsid formation (Zlotnick A et al. J. Virol. 2002, 4848).

Problems that HBV direct acting antivirals may encounter are toxicity, mutagenicity, lack of selectivity, poor efficacy, poor bioavailability, low solubility and difficulty of synthesis. There is a thus a need for additional inhibitors for the treatment, amelioration or prevention of HBV that may overcome at least one of these disadvantages or that have additional advantages such as increased potency or an increased safety window.

Administration of such therapeutic agents to an HBV infected patient, either as monotherapy or in combination with other HBV treatments or ancillary treatments, will lead to significantly reduced virus burden, improved prognosis, diminished progression of the disease and/or enhanced seroconversion rates.

### SUMMARY OF THE INVENTION

Provided herein are compounds useful for the treatment or prevention of HBV infection in a subject in need thereof, and intermediates useful in their preparation. The subject matter of the invention is a compound of Formula I

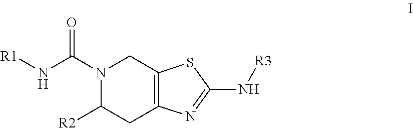

in which
R1 is phenyl or pyridyl, optionally substituted once, twice, or thrice by halo, C1-C4-alkyl, C3-C6-cycloalkyl, Cl-C4-haloalkyl or CEN
R2 is H or methyl
R3 is selected from the group comprising H, D, C1-C6-alkyl, C3-C6-cycloalkyl, C3C7-heterocycloalkyl, C2-C6-aminoalkyl, SO₂—C1-C6-alkyl, SO₂—C3-C7-cycloalkyl, SO₂—C3-C7-heterocycloalkyl, SO₂—C2-C6-hydroxyalkyl, SO₂—C2-C6-alkyl-O—C1-C6-alkyl, SO₂—C1-C4-carboxyalkyl, SO₂-aryl, SO₂-heteroaryl, SO₂—N(R12)(R13), C(═O)R4, C(═O)N(R12)(R13), C(═O)C(═O)N(R12)(R13), and C2-C6-hydroxyalkyl, optionally substituted with 1, 2, or 3 groups each independently selected from OH, halo, NH₂, acyl, SO₂CH₃, carboxy, carboxyl ester, carbamoyl, substituted carbamoyl, C6-aryl, heteroaryl, C1-C6-alkyl, C3-C6-cyclo alkyl, C3-C7-heterocyclo alkyl, C1-C6-haloalkyl, C1-C6-alkoxy, C1-C6-alkyl-O—C1-C6-alkyl, C1-C6-hydroxyalkyl, and C2-C6 alkenyloxy, preferably C1-C6-alkyl, C3-C6-cycloalkyl, C3-C7-heterocycloalkyl and C2-C6-hydroxyalkyl
R4 is selected from the group comprising C1-C6-alkyl, C1-C6-hydroxyalkyl, C1-C6-alkyl-O—C1-C6-alkyl, C3-C7-cyclo alkyl, C1-C4-carboxyalkyl, C3-C7-heterocyclo alkyl, C6-aryl, and heteroaryl optionally substituted with 1, 2, or 3 groups each independently selected from OH, halo, NH₂, acyl, SO₂CH₃, SO₃H, carboxy, carboxyl ester, carbamoyl, substituted carbamoyl, C6-aryl, heteroaryl, C1-C6-alkyl, C3-C6-cycloalkyl, C3-C7-heterocycloalkyl, C1-C6-haloalkyl, C1-C6-alkoxy, C1-C6-hydroxyalkyl, and C2-C6 alkenyloxy
R12 and R13 are independently selected from the group comprising H, C1-C6-alkyl, C2-C6-hydroxyalkyl, C2-C6-alkyl-O—C1-C6-alkyl, C3-C7-cycloalkyl, C1-C4-carboxyalkyl, C3-C7-heterocycloalkyl, C6-aryl, and heteroaryl optionally substituted with 1, 2, or 3 groups each independently selected from OH, halo, NH₂, acyl, SO₂CH₃, SO₃H, carboxy, carboxyl ester, carbamoyl, substituted carbamoyl, C6-aryl, heteroaryl, C1-C6-alkyl, C3-C6-cycloalkyl, C3-C7-heterocyclo alkyl, C1-C6-halo alkyl, C1-C6-alkoxy, C1-C6-hydroxyalkyl, and C2-C6 alkenyloxy
R12 and R13 are optionally connected to form a C3-C7-heterocycloalkyl ring containing 1 or 2 nitrogen, sulfur or oxygen atoms.

In one embodiment of the invention subject matter of the invention is a compound of Formula I in which
R1 is phenyl or pyridyl, optionally substituted once, twice, or thrice by halo, C1-C4-alkyl, C3-C6-cycloalkyl, C1-C4-haloalkyl or C≡N
R2 is H or methyl
R3 is selected from the group comprising H, D, C1-C6-alkyl, C3-C6-cycloalkyl, C3C7-heterocycloalkyl, C2-C6-aminoalkyl, SO₂—C1-C6-alkyl, SO₂—C3-C7-cycloalkyl, SO₂—C3-C7-heterocycloalkyl, SO₂—C2-C6-hydroxyalkyl, SO₂—C2-C6-alkyl-O—C1-C6-alkyl, SO₂—C1-C4-carboxyalkyl, SO₂-aryl, SO₂-heteroaryl, SO₂—N(R12)(R 13), C(═O)R4, C(═O)N(R12)(R13), C(═O)C(═O)N(R12)(R13), and C2-C6-hydroxyalkyl, optionally substituted with 1, 2, or 3 groups each independently selected from OH, halo, NH₂, acyl, SO₂CH₃, carboxy, carboxyl ester, carbamoyl, substituted carbamoyl, C6-aryl, heteroaryl, C1-C6-alkyl, C3-C6-cyclo alkyl, C3-C7-heterocyclo alkyl, C1-C6-haloalkyl, C1-C6-alkoxy, C1-C6-alkyl-O—C1-C6-alkyl, C1-C6-hydroxyalkyl, and C2-C6 alkenyloxy, preferably C1-C6-alkyl, C3-C6-cycloalkyl, C3-C7-heterocycloalkyl and C2-C6-hydroxyalkyl
R4 is selected from the group comprising C1-C6-alkyl, C1-C6-hydroxyalkyl, C1-C6-alkyl-O—C1-C6-alkyl, C3-C7-cycloalkyl, C1-C4-carboxyalkyl, C3-C7-heterocyclo alkyl, C6-aryl, heteroaryl optionally substituted with 1, 2, or 3 groups each independently selected from OH, halo, NH₂, acyl, SO₂CH₃, SO₃H, carboxy, carboxyl ester, carbamoyl, substituted carbamoyl, C6-aryl, heteroaryl, C1-C6-alkyl, C3-C6-cycloalkyl, C3-C7-heterocycloalkyl, C1-C6-haloalkyl, C1-C6-alkoxy, C1-C6-hydroxyalkyl, and C2-C6 alkenyloxy
R12 and R13 are independently selected from the group comprising H, C1-C6-alkyl, C2-C6-hydroxyalkyl, C2-C6-alkyl-O—C1-C6-alkyl, C3-C7-cycloalkyl, C1-C4-carboxyalkyl, C3-C7-heterocycloalkyl, C6-aryl, and heteroaryl optionally substituted with 1, 2, or 3 groups each independently selected from OH, halo, NH₂, acyl, SO₂CH₃, SO₃H, carboxy, carboxyl ester, carbamoyl, substituted carbamoyl, C6-aryl, heteroaryl, C1-C6-alkyl, C3-C6-cycloalkyl, C3-C7-heterocyclo alkyl, C1-C6-haloalkyl, C1-C6-alkoxy, C1-C6-hydroxyalkyl, and C2-C6 alkenyloxy.

In one embodiment subject matter of the present invention is a compound according to Formula I in which R1 is phenyl or pyridyl, optionally substituted once, twice, or thrice by halo, C1-C4-alkyl, C3-C6-cycloalkyl, C1-C4-haloalkyl or C≡N.

In one embodiment subject matter of the present invention is a compound according to Formula I in which R2 is H or methyl.

In one embodiment subject matter of the present invention is a compound according to Formula I in which R3 is selected from the group comprising H, D, C1-C6-alkyl, C3-C6-cycloalkyl, C3C7-heterocycloalkyl, C2-C6-aminoalkyl, SO₂-C1-C6-alkyl, SO₂-C3-C7-cycloalkyl, SO₂—C3-C7-heterocycloalkyl, SO₂-C2-C6-hydroxyalkyl, SO₂—C2-C6-alkyl-O—C1-C6-alkyl, SO₂—C1-C4-carboxyalkyl, SO₂-aryl, SO₂-heteroaryl, SO₂—N(R12)(R13), C(═O)R4, C(═O)N(R12)(R13), C(═O)C(═O)N(R12)(R13), and C2-C6-hydroxyalkyl, optionally substituted with 1, 2, or 3 groups each independently selected from OH, halo, NH₂, acyl, SO₂CH₃, carboxy, carboxyl ester, carbamoyl, substituted carbamoyl, C6-aryl, heteroaryl, C1-C6-alkyl, C3-C6-cycloalkyl, C3-C7-heterocycloalkyl, C1-C6-haloalkyl, C1-C6-alkoxy, C1-C6-alkyl-O—C1-C6-alkyl, C1-C6-hydroxyalkyl, and C2-C6 alkenyloxy, preferably C1-C6-alkyl, C3-C6-cycloalkyl, C3-C7-heterocycloalkyl and C2-C6-hydroxyalkyl.

In one embodiment subject matter of the present invention is a compound according to Formula I in which R4 is selected from the group comprising C1-C6-alkyl, C1-C6-hydroxyalkyl, C1-C6-alkyl-O—C1-C6-alkyl, C3-C7-cycloalkyl, C1-C4-carboxyalkyl, C3-C7-heterocycloalkyl, C6-aryl, and heteroaryl optionally substituted with 1, 2, or 3 groups each independently selected from OH, halo, NH₂, acyl, SO₂CH₃, SO₃H, carboxy, carboxyl ester, carbamoyl, substituted carbamoyl, C6-aryl, heteroaryl, C1-C6-alkyl, C3-C6-cycloalkyl, C3-C7-heterocycloalkyl, C1-C6-haloalkyl, C1-C6-alkoxy, C1-C6-hydroxyalkyl, and C2-C6 alkenyloxy.

In one embodiment subject matter of the present invention is a compound according to Formula I in which R12 and R13 are selected from the group comprising H, C1-C6-alkyl, C2-C6-hydroxyalkyl, C2-C6-alkyl-O—C1-C6-alkyl, C3-C7-cycloalkyl, C1-C4-carboxyalkyl, C3-C7-heterocycloalkyl, C6-aryl, and heteroaryl optionally substituted with 1, 2, or 3 groups each independently selected from OH, halo, NH₂, acyl, SO₂CH₃, SO₃H, carboxy, carboxyl ester, carbamoyl, substituted carbamoyl, C6-aryl, heteroaryl, C1-C6-alkyl, C3-C6-cycloalkyl, C3-C7-heterocycloalkyl, C1-C6-haloalkyl, C1-C6-alkoxy, C1-C6-hydroxyalkyl, and C2-C6 alkenyloxy.

In one embodiment subject matter of the present invention is a compound according to Formula I in which R12 and R13 are optionally connected to form a C3-C7-heterocycloalkyl ring containing 1 or 2 nitrogen, sulfur or oxygen atoms.

One embodiment of the invention is a compound of Formula I or a pharmaceutically acceptable salt thereof according to the invention, for use in the prevention or treatment of an HBV infection in subject.

One embodiment of the invention is a pharmaceutical composition comprising a compound of Formula I or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula I or a pharmaceutically acceptable salt thereof according to the present invention.

A further embodiment of the invention is a compound of Formula II or a pharmaceutically acceptable salt thereof according to the invention, for use in the prevention or treatment of an HBV infection in subject in need thereof

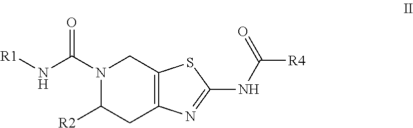

in which
R1 is phenyl or pyridyl, optionally substituted once, twice, or thrice by halo, C1-C4-alkyl, C3-C6-cycloalkyl, C1-C4-haloalkyl or C≡N
R2 is H or methyl
R4 is selected from the group comprising C1-C6-alkyl, C1-C6-hydroxyalkyl, C1-C6-alkyl-O—C1-C6-alkyl, C3-C7-cycloalkyl, C1-C4-carboxyalkyl, C3-C7-heterocyclo alkyl, C6-aryl, and heteroaryl optionally substituted with 1, 2, or 3 groups each independently selected from OH, halo, NH₂, acyl, SO₂CH₃, SO₃H, carboxy, carboxyl ester, carbamoyl, substituted carbamoyl, C6-aryl, heteroaryl, C1-C6-alkyl, C3-C6-cycloalkyl, C3-C7-heterocycloalkyl, C1-C6-haloalkyl, C1-C6-alkoxy, C2-C6-hydroxyalkyl, and C2-C6 alkenyloxy.

In one embodiment subject matter of the present invention is a compound according to Formula II in which R1 is phenyl or pyridyl, optionally substituted once, twice, or thrice by halo, C1-C4-alkyl, C3-C6-cycloalkyl, C1-C4-haloalkyl or C≡N.

In one embodiment subject matter of the present invention is a compound according to Formula II in which R2 is H or methyl.

In one embodiment subject matter of the present invention is a compound according to Formula II in which R4 is C1-C6-alkyl, C1-C6-hydroxyalkyl, C1-C6-alkyl-O—C1-C6-alkyl, C3-C7-cycloalkyl, C1-C4-carboxyalkyl, C3-C7-heterocycloalkyl, C6-aryl, or heteroaryl optionally substituted with 1, 2, or 3 groups each independently selected from OH, halo, NH₂, acyl, SO₂CH₃, SO₃H, carboxy, carboxyl ester, carbamoyl, substituted carbamoyl, C6-aryl, heteroaryl, C1-C6-alkyl, C3-C6-cycloalkyl, C3-C7-heterocycloalkyl, C1-C6-haloalkyl, C1-C6-alkoxy, C2-C6-hydroxyalkyl, and C2-C6 alkenyloxy.

One embodiment of the invention is a compound of Formula II or a pharmaceutically acceptable salt thereof according to the invention, for use in the prevention or treatment of an HBV infection in subject.

One embodiment of the invention is a pharmaceutical composition comprising a compound of Formula II or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula II or a pharmaceutically acceptable salt thereof according to the present invention.

A further embodiment of the invention is a compound of Formula III or a pharmaceutically acceptable salt thereof according to the invention, for use in the prevention or treatment of an HBV infection in subject in need thereof.

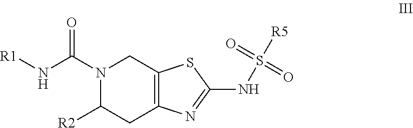

in which
R1 is phenyl or pyridyl, optionally substituted once, twice, or thrice by halo, C1-C4-alkyl, C3-C6-cycloalkyl, C1-C4-haloalkyl or C≡N
R2 is H or methyl
R5 is selected from the group comprising C1-C6-alkyl, C2-C6-hydroxyalkyl, C2-C6-alkyl-O—C1-C6-alkyl, C3-C7-cycloalkyl, C1-C4-carboxyalkyl, C3-C7-heterocycloalkyl, C6-aryl, and heteroaryl, optionally substituted with 1, 2, or 3 groups each independently selected from OH, halo, NH₂, acyl, SO₂CH₃, SO₃H, carboxy, carboxyl ester, carbamoyl, substituted carbamoyl, C6-aryl, heteroaryl, C1-C6-alkyl, C3-C6-cycloalkyl, C3-C7-heterocycloalkyl, C1-C6-haloalkyl, C1-C6-alkoxy, C1-C6-hydroxyalkyl, and C2-C6 alkenyloxy.

In one embodiment subject matter of the present invention is a compound according to Formula III in which R1 is phenyl or pyridyl, optionally substituted once, twice, or thrice by halo, C1-C4-alkyl, C3-C6-cycloalkyl, C1-C4-haloalkyl or C≡N.

In one embodiment subject matter of the present invention is a compound according to Formula III in which R2 is H or methyl.

In one embodiment subject matter of the present invention is a compound according to Formula III in which R5 is C1-C6-alkyl, C2-C6-hydroxyalkyl, C2-C6-alkyl-O—C1-C6-alkyl, C3-C7-cycloalkyl, C1-C4-carboxyalkyl, C3-C7-heterocycloalkyl, C6-aryl, or heteroaryl, optionally substituted with 1, 2, or 3 groups each independently selected from OH, halo, NH₂, acyl, SO₂CH₃, SO₃H, carboxy, carboxyl ester, carbamoyl, substituted carbamoyl, C6-aryl, heteroaryl, C1-C6-alkyl, C3-C6-cycloalkyl, C3-C7-heterocycloalkyl, C1-C6-haloalkyl, C1-C6-alkoxy, C1-C6-hydroxyalkyl, and C2-C6 alkenyloxy.

One embodiment of the invention is a compound of Formula III or a pharmaceutically acceptable salt thereof according to the invention, for use in the prevention or treatment of an HBV infection in subject.

One embodiment of the invention is a pharmaceutical composition comprising a compound of Formula III or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula III or a pharmaceutically acceptable salt thereof according to the present invention.

A further embodiment of the invention is a compound of Formula IV or a pharmaceutically acceptable salt thereof according to the invention, for use in the prevention or treatment of an HBV infection in subject in need thereof.

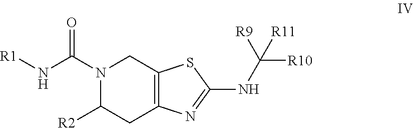

in which
R1 is phenyl or pyridyl, optionally substituted once, twice, or thrice by halo, C1-C4-alkyl, C3-C6-cycloalkyl, C1-C4-haloalkyl or C≡N.
R2 is H or methyl
R9, R10 and R11 are independently selected from the group comprising H, C1-C5-hydroxyalkyl, C1-C5-alkyl-O—C1-C6-alkyl, C1-C5-alkyl, C3-C7-cycloalkyl, C1-C3-carboxyalkyl, C3-C7-heterocycloalkyl, C6-aryl, and heteroaryl, wherein C1-C5-alkyl, C1-C5-hydroxyalkyl, C1-C5-alkyl-O—C1-C6-alkyl and C1-C3-carboxyalkyl are optionally substituted with 1, 2, or 3 groups each independently selected from OH, halo, NH₂, acyl, SO₂CH₃, SO₃H, carboxy, carboxyl ester, carbamoyl, substituted carbamoyl, C6-aryl, heteroaryl, C1-C6-alkyl, C3-C6-cycloalkyl, C3-C7-heterocycloalkyl, C1-C6-haloalkyl, C1-C6-alkoxy, C1-C6-hydroxyalkyl, and C2-C6 alkenyloxy
R9 and R10 are optionally connected to form a C3-C7 cycloalkyl ring, or a C4-C7-heterocycloalkyl ring containing 1 or 2 nitrogen, sulfur or oxygen atoms.

In one embodiment subject matter of the present invention is a compound according to Formula IV in which R1 is phenyl or pyridyl, optionally substituted once, twice, or thrice by halo, C1-C4-alkyl, C3-C6-cycloalkyl, C1-C4-haloalkyl or C≡N.

In one embodiment subject matter of the present invention is a compound according to Formula IV in which R2 is selected from the group comprising H and methyl.

In one embodiment subject matter of the present invention is a compound according to Formula IV in which R9, R10 and R11 are independently selected from the group comprising H, C1-C5-hydroxyalkyl, C1-C5-alkyl-O—C1-C6-alkyl, C1-C5-alkyl, C3-C7-cycloalkyl, C1-C3-carboxyalkyl, C3-C7-heterocycloalkyl, C6-aryl, and heteroaryl, wherein C1-C5-alkyl, C1-C5-hydroxyalkyl, C1-C5-alkyl-O—C1-C6-alkyl and C1-C3-carboxyalkyl are optionally substituted with 1, 2, or 3 groups each independently selected from OH, halo, NH_{2,} acyl, SO₂CH₃, SO₃H, carboxy, carboxyl ester, carbamoyl, substituted carbamoyl, C6-aryl, heteroaryl, C1-C6-alkyl, C3-C6-cycloalkyl, C3-C7-heterocycloalkyl, C1-C6-halo alkyl, C1-C6-alkoxy, C1-C6-hydroxyalkyl, and C2-C6 alkenyloxy.

In one embodiment subject matter of the invention is a compound according to Formula IV in which R9 and R10 are optionally connected to form a C3-C7 cycloalkyl ring, or a C4-C7-heterocycloalkyl ring containing 1 or 2 nitrogen, sulfur or oxygen atoms.

One embodiment of the invention is a compound of Formula IV or a pharmaceutically acceptable salt thereof according to the invention, for use in the prevention or treatment of an HBV infection in subject.

One embodiment of the invention is a pharmaceutical composition comprising a compound of Formula IV or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula IV or a pharmaceutically acceptable salt thereof according to the present invention.

In some embodiments, the dose of a compound of the invention is from about 1 mg to about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., another drug for HBV treatment) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof. All before mentioned doses refer to daily doses per patient.

In general it is contemplated that an antiviral effective daily amount would be from about 0.01 to about 50 mg/kg, or about 0.01 to about 30 mg/kg body weight. It maybe appropriate to administer the required dose as two, three, four or more sub-doses at appropriate intervals throughout the day. Said sub-doses may be formulated as unit dosage forms, for example containing about 1 to about 500 mg, or about 1 to about 300 mg or about 1 to about 100 mg, or about 2 to about 50 mg of active ingredient per unit dosage form.

The compounds of the invention may, depending on their structure, exist as salts, solvates or hydrates. The invention therefore also encompasses the salts, solvates or hydrates and respective mixtures thereof.

The compounds of the invention may, depending on their structure, exist in tautomeric or stereoisomeric forms (enantiomers, diastereomers). The invention therefore also encompasses the tautomers, enantiomers or diastereomers and respective mixtures thereof. The stereoisomerically uniform constituents can be isolated in a known manner from such mixtures of enantiomers and/or diastereomers.

Definitions

Listed below are definitions of various terms used to describe this invention. These definitions apply to the terms as they are used throughout this specification and claims unless otherwise limited in specific instances either individually or as part of a larger group.

Unless defined otherwise all technical and scientific terms used herein generally have the same meaning as commonly understood by one of ordinary skill in the art to which this invention belongs. Generally the nomenclature used herein and the laboratory procedures in cell culture, molecular genetics, organic chemistry and peptide chemistry are those well known and commonly employed in the art.

As used herein the articles “a” and “an” refer to one or to more than one (i.e. to at least one) of the grammatical object of the article. By way of example, “an element” means one element or more than one element. Furthermore, use of the term “including” as well as other forms such as “include”, “includes” and “included”, is not limiting.

As used herein the term “capsid assembly modulator” refers to a compound that disrupts or accelerates or inhibits or hinders or delays or reduces or modifies normal capsid assembly (e.g. during maturation) or normal capsid disassembly (e.g. during infectivity) or perturbs capsid stability, thereby inducing aberrant capsid morphology or aberrant capsid function. In one embodiment, a capsid assembly modulator accelerates capsid assembly or disassembly thereby inducing aberrant capsid morphology. In another embodiment a capsid assembly modulator interacts (e.g. binds at an active site, binds at an allosteric site or modifies and/or hinders folding and the like), with the major capsid assembly protein (HBV-CP), thereby disrupting capsid assembly or disassembly. In yet another embodiment a capsid assembly modulator causes a perturbation in the structure or function of HBV-CP (e.g. the ability of HBV-CP to assemble, disassemble, bind to a substrate, fold into a suitable conformation or the like which attenuates viral infectivity and/or is lethal to the virus).

As used herein the term “treatment” or “treating” is defined as the application or administration of a therapeutic agent i.e., a compound of the invention (alone or in combination with another pharmaceutical agent) to a patient, or application or administration of a therapeutic agent to an isolated tissue or cell line from a patient (e.g. for diagnosis or ex vivo applications) who has an HBV infection, a symptom of HBV infection, or the potential to develop an HBV infection with the purpose to cure, heal, alleviate, relieve, alter, remedy, ameliorate, improve or affect the HBV infection, the symptoms of HBV infection or the potential to develop an HBV infection. Such treatments may be specifically tailored or modified based on knowledge obtained from the field of pharmacogenomics.

As used herein the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein the term “patient”, “individual” or “subject” refers to a human or a non-human mammal. Non-human mammals include for example livestock and pets such as ovine, bovine, porcine, feline, and murine mammals. Preferably the patient, subject, or individual is human.

As used herein the terms “effective amount”, “pharmaceutically effective amount”, and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein the term “pharmaceutically acceptable” refers to a material such as a carrier or diluent which does not abrogate the biological activity or properties of the compound and is relatively non-toxic i.e. the material may be administered to an individual without causing undesirable biological effects or interacting in a deleterious manner with any of the components of the composition in which it is contained.

As used herein the term “pharmaceutically acceptable salt” refers to derivatives of the disclosed compounds wherein the parent compound is modified by converting an existing acid or base moiety to its salt form. Examples of pharmaceutically acceptable salts include but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent or in a mixture of the two; generally nonaqueous media like ether, ethyl acetate, ethanol, isopropanol, or acetonitrile are preferred. Lists of suitable salts are found in Remington's Pharmaceutical Sciences 17^{th} ed. Mack Publishing Company, Easton, Pa., 1985 p.1418 and Journal of Pharmaceutical Science, 66, 2 (1977), each of which is incorporated herein by reference in its entirety.

As used herein the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including but not limited to intravenous, oral, aerosol, rectal, parenteral, ophthalmic, pulmonary and topical administration.

As used herein the term “pharmaceutically acceptable carrier” means a pharmaceutically acceptable material, composition or carrier such as a liquid or solid filler, stabilizer, dispersing agent, suspending agent, diluent, excipient, thickening agent, solvent or encapsulating material involved in carrying or transporting a compound useful within the invention within or to the patient such that it may perform its intended function. Typically such constructs are carried or transported from one organ, or portion of the body, to another organ or portion of the body. Each carrier must be “acceptable” in the sense of being compatible with the other ingredients of the formulation including the compound use within the invention and not injurious to the patient. Some examples of materials that may serve as pharmaceutically acceptable carriers include: sugars, such as lactose, glucose and sucrose; starches such as corn starch and potato starch; cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols such as propylene glycol; polyols such as glycerin, sorbitol, mannitol and polyethylene glycol; esters such as ethyl oleate and ethyl laurate; agar; buffering agents, such as magnesium hydroxide and aluminium hydroxide; surface active agents; alginic acid; pyrogen-free water; isotonic saline; Ringer's solution; ethyl alcohol; phosphate buffer solutions and other non-toxic compatible substances employed in pharmaceutical formulations.

As used herein “pharmaceutically acceptable carrier” also includes any and all coatings, antibacterial and antifungal agents and absorption delaying agents and the like that are compatible with the activity of the compound useful within the invention and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described for example in Remington's Pharmaceutical Sciences (Genaro, Ed., Mack Publishing Company, Easton, Pa., 1985) which is incorporated herein by reference.

As used herein, the term “substituted” means that an atom or group of atoms has replaced hydrogen as the substituent attached to another group.

As used herein, the term “comprising” also encompasses the option “consisting of”.

As used herein, the term “alkyl” by itself or as part of another substituent means, unless otherwise stated, a straight or branched chain hydrocarbon having the number of carbon atoms designated (i.e. C1-C6-alkyl means one to six carbon atoms) and includes straight and branched chains. Examples include methyl, ethyl, propyl, isopropyl, butyl, isobutyl, tert-butyl, pentyl, neopentyl, and hexyl. In addition, the term “alkyl” by itself or as part of another substituent can also mean a C1-C3 straight chain hydrocarbon substituted with a C3-C5-carbocylic ring. Examples include (cyclopropyl)methyl, (cyclobutyl)methyl and (cyclopentyl)methyl. For the avoidance of doubt, where two alkyl moieties are present in a group, the alkyl moieties may be the same or different.

As used herein the term “alkenyl” denotes a monovalent group derived from a hydrocarbon moiety containing at least two carbon atoms and at least one carbon-carbon double bond of either E or Z stereochemistry. The double bond may or may not be the point of attachment to another group.

Alkenyl groups (e.g. C2-C8-alkenyl) include, but are not limited to for example ethenyl, propenyl, prop-1-en-2-yl, butenyl, methyl-2-buten-1-yl, heptenyl and octenyl. For the avoidance of doubt, where two alkenyl moieties are present in a group, the alkyl moieties may be the same or different.

As used herein, a C2-C6-alkynyl group or moiety is a linear or branched alkynyl group or moiety containing from 2 to 6 carbon atoms, for example a C2-C4 alkynyl group or moiety containing from 2 to 4 carbon atoms. Exemplary alkynyl groups include —C≡CH or —CH₂—C≡C, as well as 1- and 2-butynyl, 2-pentynyl, 3-pentynyl, 4-pentynyl, 2-hexynyl, 3-hexynyl, 4-hexynyl and 5-hexynyl. For the avoidance of doubt, where two alkynyl moieties are present in a group, they may be the same or different.

As used herein, the term “halo” or “halogen” alone or as part of another substituent means unless otherwise stated a fluorine, chlorine, bromine, or iodine atom, preferably fluorine, chlorine, or bromine, more preferably fluorine or chlorine. For the avoidance of doubt, where two halo moieties are present in a group, they may be the same or different.

As used herein, a C1-C6-alkoxy group or C2-C6-alkenyloxy group is typically a said C1-C6-alkyl (e.g. a C1-C4 alkyl) group or a said C2-C6-alkenyl (e.g. a C2-4 alkenyl) group respectively which is attached to an oxygen atom.

As used herein the term “aryl” employed alone or in combination with other terms, means unless otherwise stated a carbocyclic aromatic system containing one or more rings (typically one, two or three rings) wherein such rings may be attached together in a pendant manner such as a biphenyl, or may be fused, such as naphthalene. Examples of aryl groups include phenyl, anthracyl, and naphthyl. Preferred examples are phenyl (e.g. C6-aryl) and biphenyl (e.g. C12-aryl). In some embodiments aryl groups have from six to sixteen carbon atoms. In some embodiments aryl groups have from six to twelve carbon atoms (e.g. C6-C12-aryl). In some embodiments, aryl groups have six carbon atoms (e.g. C6-aryl).

As used herein the terms “heteroaryl” and “heteroaromatic” refer to a heterocycle having aromatic character containing one or more rings (typically one, two or three rings). Heteroaryl substituents may be defined by the number of carbon atoms e.g. C1-C9-heteroaryl indicates the number of carbon atoms contained in the heteroaryl group without including the number of heteroatoms. For example a C1-C9-heteroaryl will include an additional one to four heteroatoms. A polycyclic heteroaryl may include one or more rings that are partially saturated. Non-limiting examples of heteroaryls include:

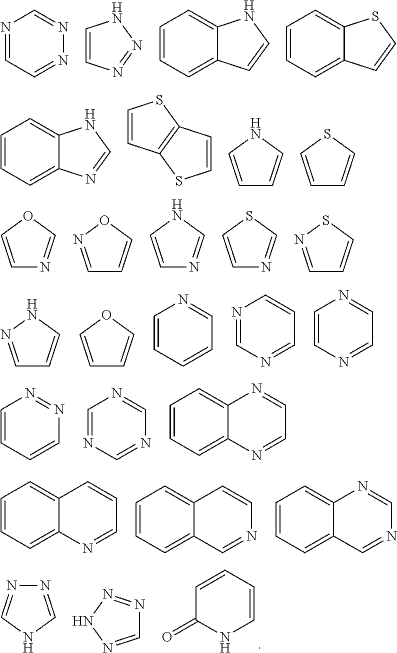

Additional non-limiting examples of heteroaryl groups include pyridyl, pyrazinyl, pyrimidinyl (including e.g. 2-and 4-pyrimidinyl), pyridazinyl, thienyl, furyl, pyrrolyl (including e.g., 2-pyrrolyl), imidazolyl, thiazolyl, oxazolyl, pyrazolyl (including e.g. 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyland 1,3,4-oxadiazolyl. Non-limiting examples of polycyclic heterocycles and heteroaryls include indolyl (including 3-, 4-, 5-, 6-and 7-indolyl), indolinyl, quinolyl, tetrahydroquinolyl, isoquinolyl (including, e.g. 1-and 5-isoquinolyl), 1,2,3,4-tetrahydroisoquinolyl, cinnolinyl, quinoxalinyl (including, e.g 2-and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (including, e .g. 3-, 4-, 5-, 6-, and 7-benzofuryl), 2,3-dihydrobenzofuryl, 1,2-benzisoxazolyl, benzothienyl (including e.g. 3-, 4-, 5-, 6-, and 7-benzothienyl), benzoxazolyl, benzothiazolyl (including e.g. 2-benzothiazolyl and 5-benzothiazolyl), purinyl, benzimidazolyl (including e.g., 2-benzimidazolyl), benzotriazolyl, thioxanthinyl, carbazolyl, carbolinyl, acridinyl, pyrrolizidinyl and quinolizidinyl.

As used herein the term “haloalkyl” is typically a said alkyl, alkenyl, alkoxy or alkenoxy group respectively wherein any one or more of the carbon atoms is substituted with one or more said halo atoms as defined above. Haloalkyl embraces monohaloalkyl, dihaloalkyl, and polyhaloalkyl radicals. The term “haloalkyl”includes but is not limited to fluoromethyl, 1-fluoroethyl, difluoromethyl, 2,2-difluoroethyl, 2,2,2-trifluoroethyl, trifluoromethyl, chloromethyl, dichloromethyl, trichloromethyl, pentafluoroethyl, difluoromethoxy, and trifluoromethoxy.

As used herein, a C1-C6-hydroxyalkyl group is a said C1-C6 alkyl group substituted by one or more hydroxy groups. Typically, it is substituted by one, two or three hydroxyl groups. Preferably, it is substituted by a single hydroxy group.

As used herein, a C1-C6-aminoalkyl group is a said C1-C6 alkyl group substituted by one or more amino groups. Typically, it is substituted by one, two or three amino groups. Preferably, it is substituted by a single amino group.

As used herein, a C1-C4-carboxyalkyl group is a said C1-C4 alkyl group substituted by carboxyl group.

As used herein, a C1-C4-carboxamidoalkyl group is a said C1-C4 alkyl group substituted by a substituted or unsubstituted carboxamide group.

As used herein, a C1-C4-acylsulfonamido-alkyl group is a said C1-C4 alkyl group substituted by an acylsulfonamide group of general formula C(═O)NHSO₂CH₃ or C(═O)NHSO₂-c-Pr.

As used herein the term “cycloalkyl” refers to a monocyclic or polycyclic nonaromatic group wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having 3 to 10 ring atoms (C3-C10-cycloalkyl), groups having 3 to 8 ring atoms (C3-C8-cycloalkyl), groups having 3 to 7 ring atoms (C3-C7-cycloalkyl) and groups having 3 to 6 ring atoms (C3-C6-cycloalkyl). Illustrative examples of cycloalkyl groups include, but are not limited to the following moieties:

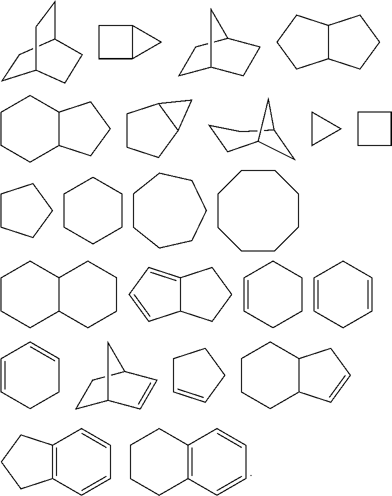

Monocyclic cycloalkyls include but are not limited to cyclopropyl, cyclobutyl, cyclopentyl, cyclohexyl, cycloheptyl, and cyclooctyl. Dicyclic cycloalkyls include but are not limited to tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups both of which refer to a nonaromatic carbocycle as defined herein which contains at least one carbon-carbon double bond or one carbon-carbon triple bond.

As used herein the terms “heterocycloalkyl” and “heterocyclyl” refer to a heteroalicyclic group containing one or more rings (typically one, two or three rings), that contains one to four ring heteroatoms each selected from oxygen, sulfur and nitrogen. In one embodiment each heterocyclyl group has from 3 to 10 atoms in its ring system with the proviso that the ring of said group does not contain two adjacent oxygen or sulfur atoms. In one embodiment each heterocyclyl group has a fused bicyclic ring system with 3 to 10 atoms in the ring system, again with the proviso that the ring of said group does not contain two adjacent oxygen or sulfur atoms. In one embodiment each heterocyclyl group has a bridged bicyclic ring system with 3 to 10 atoms in the ring system, again with the proviso that the ring of said group does not contain two adjacent oxygen or sulfur atoms. In one embodiment each heterocyclyl group has a spiro-bicyclic ring system with 3 to 10 atoms in the ring system, again with the proviso that the ring of said group does not contain two adjacent oxygen or sulfur atoms. Heterocyclyl substituents may be alternatively defined by the number of carbon atoms e.g. C2-C8-heterocyclyl indicates the number of carbon atoms contained in the heterocyclic group without including the number of heteroatoms. For example a C2-C8-heterocyclyl will include an additional one to four heteroatoms. In another embodiment the heterocycloalkyl group is fused with an aromatic ring. In another embodiment the heterocycloalkyl group is fused with a heteroaryl ring. In one embodiment the nitrogen and sulfur heteroatoms may be optionally oxidized and the nitrogen atom may be optionally quaternized. The heterocyclic system may be attached, unless otherwise stated, at any heteroatom or carbon atom that affords a stable structure. An example of a 3-membered heterocyclyl group includes and is not limited to aziridine. Examples of 4-membered heterocycloalkyl groups include, and are not limited to azetidine and a beta-lactam. Examples of 5-membered heterocyclyl groups include, and are not limited to pyrrolidine, oxazolidine and thiazolidinedione. Examples of 6-membered heterocycloalkyl groups include, and are not limited to, piperidine, morpholine, piperazine, N-acetylpiperazine and N-acetylmorpholine. Other non-limiting examples of heterocyclyl groups are

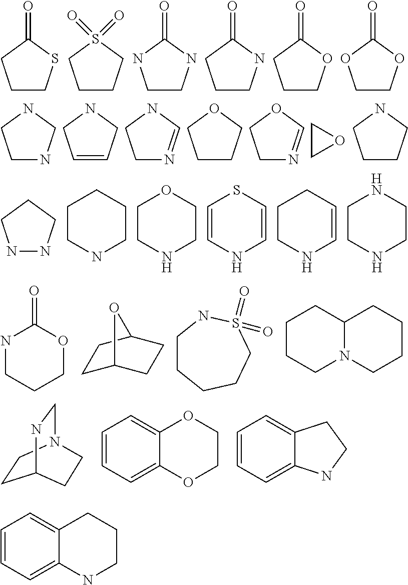

Examples of heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, 1,3-dioxolane, homopiperazine, homopiperidine, 1,3-dioxepane, 47-dihydro-1,3-dioxepin, and hexamethyleneoxide.

As used herein, the term “aromatic” refers to a carbocycle or heterocycle with one or more polyunsaturated rings and having aromatic character i.e. having (4n+2) delocalized π(pi) electrons where n is an integer.

As used herein, the term “acyl”, employed alone or in combination with other terms, means, unless otherwise stated, to mean to an alkyl, cycloalkyl, heterocycloalkyl, aryl or heteroaryl group linked via a carbonyl group.

As used herein, the terms “carbamoyl” and “substituted carbamoyl”, employed alone or in combination with other terms, means, unless otherwise stated, to mean a carbonyl group linked to an amino group optionally mono or di-substituted by hydrogen, alkyl, cycloalkyl, heterocycloalkyl, aryl or heteroaryl. In some embodiments, the nitrogen substituents will be connected to form a heterocyclyl ring as defined above.

As used herein, the term “carboxy” and by itself or as part of another substituent means, unless otherwise stated, a group of formula C(═O)OH.

As used herein, the term “carboxyl ester” by itself or as part of another substituent means, unless otherwise stated, a group of formula C(═O)OX, wherein X is selected from the group consisting of C1-C6-alkyl, C3-C7-cycloalkyl, and aryl.

As used herein the term “prodrug” represents a derivative of a compound of Formula I or Formula II or Formula III or Formula IV which is administered in a form which, once administered, is metabolised in vivo into an active metabolite also of Formula I or Formula II or Formula III or Formula IV.

Various forms of prodrug are known in the art. For examples of such prodrugs see: Design of Prodrugs, edited by H. Bundgaard, (Elsevier, 1985) and Methods in Enzymology, Vol. 42, p. 309-396, edited by K. Widder, et al. (Academic Press, 1985); A Textbook of Drug Design and Development, edited by Krogsgaard-Larsen and H. Bundgaard, Chapter 5 “Design and Application of Prodrugs” by H. Bundgaard p. 113-191 (1991); H. Bundgaard, Advanced Drug Delivery Reviews 8, 1-38 (1992); H. Bundgaard, et al., Journal of Pharmaceutical Sciences, 77, 285 (1988); and N. Kakeya, et al., Chem. Pharm. Bull., 32, 692 (1984).

Examples of prodrugs include cleavable esters of compounds of Formula I or Formula II or Formula III or Formula IV. An in vivo cleavable ester of a compound of the invention containing a carboxy group is, for example, a pharmaceutically acceptable ester which is cleaved in the human or animal body to produce the parent acid. Suitable pharmaceutically acceptable esters for carboxy include C1-C6-alkyl ester, for example methyl or ethyl esters; C1-C6 alkoxymethyl esters, for example methoxymethyl ester; C1-C6 acyloxymethyl esters; phthalidyl esters; C3-C8 cycloalkoxyc arbonyloxyC1-C6-alkyl esters, for example 1-cyclohexylc arbonyloxyethyl; 1-3-dioxolan-2-ylmethylesters, for example 5-methyl-1,3-dioxolan-2-ylmethyl; C1-C6 alkoxycarbonyloxyethyl esters, for example 1-methoxycarbonyloxyethyl; aminocarbonylmethyl esters and mono-or di-N—(C1-C6-alkyl) versions thereof, for example N, N-dimethylaminocarbonylmethyl esters and N-ethylaminocarbonylmethyl esters; and may be formed at any carboxy group in the compounds of the invention.

An in vivo cleavable ester of a compound of the invention containing a hydroxy group is, for example, a pharmaceutically-acceptable ester which is cleaved in the human or animal body to produce the parent hydroxy group. Suitable pharmaceutically acceptable esters for hydroxy include C1-C6-acyl esters, for example acetyl esters; and benzoyl esters wherein the phenyl group may be substituted with aminomethyl or N-substituted mono-or di-C1-C6-alkyl aminomethyl, for example 4-aminomethylbenzoyl esters and 4-N,N-dimethylaminomethylbenzoyl esters.

Preferred prodrugs of the invention include acetyloxy and carbonate derivatives. For example, a hydroxy group of a compound of Formula I or Formula II or Formula III or Formula IV can be present in a prodrug as —O—CORⁱ or —O—C(O)ORⁱ where Rⁱ is unsubstituted or substituted C1-C4 alkyl. Substituents on the alkyl groups are as defined earlier. Preferably the alkyl groups in Rⁱ is unsubstituted, preferable methyl, ethyl, isopropyl or cyclopropyl.

Other preferred prodrugs of the invention include amino acid derivatives. Suitable amino acids include α-amino acids linked to compounds of Formula I or Formula II or Formula III or Formula IV via their C(O)OH group. Such prodrugs cleave in vivo to produce compounds of Formula I or Formula II or Formula III or Formula IV bearing a hydroxy group. Accordingly such amino acid groups are preferably employed positions of Formula I or Formula II or Formula III or Formula IV where a hydroxy group is eventually required. Exemplary prodrugs of this embodiment of the invention are therefore compounds of Formula I or Formula II or Formula III or Formula IV bearing a group of Formula —OC(O)—CH(NH₂)Rⁱⁱ where Rⁱⁱ is an amino acid side chain. Preferred amino acids include glycine, alanine, valine and serine. The amino acid can also be functionalised, for example the amino group can be alkylated. A suitable functionalised amino acid is N,N-dimethylglycine. Preferably the amino acid is valine.

Other preferred prodrugs of the invention include phosphoramidate derivatives. Various forms of phosphoramidate prodrugs are known in the art. For example of such prodrugs see Serpi et al., Curr. Protoc. Nucleic Acid Chem. 2013, Chapter 15, Unit 15.5 and Mehellou et al., Chem Med Chem, 2009, 4 pp. 1779-1791. Suitable phosphoramidates include (phenoxy)-α-amino acids linked to compounds of Formula I or Formula II or Formula III or Formula IV via their —OH group. Such prodrugs cleave in vivo to produce compounds of Formula I bearing a hydroxy group. Accordingly, such phosphoramidate groups are preferably employed positions of Formula I or Formula II or Formula III or Formula IV where a hydroxy group is eventually required. Exemplary prodrugs of this embodiment of the invention are therefore compounds of Formula I or Formula II or Formula III or Formula IV bearing a group of Formula —OP(O)(ORⁱⁱⁱ)R^{iv} where Rⁱⁱⁱ is alkyl, cycloalkyl, aryl or heteroaryl, and R^{iv} is a group of Formula —NH—CH(R^{v})C(O)OR^{vi}, wherein R^{v} is an amino acid side chain and R^{vi} is alkyl, cycloalkyl, aryl or heterocyclyl. Preferred amino acids include glycine, alanine, valine and serine. Preferably the amino acid is alanine. R^{v} is preferably alkyl, most preferably isopropyl.

Subject matter of the present invention is also a method of preparing the compounds of the present invention. Subject matter of the invention is, thus, a method for the preparation of a compound of Formula I according to the present invention by reacting a compound of Formula V

R1-N═C═O V

in which R1 is above-defined, with a compound of Formula VI

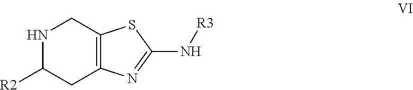

in which R2, and R3 are as above-defined.

### EXAMPLES

The invention is now described with reference to the following Examples. These Examples are provided for the purpose of illustration only, and the invention is not limited to these Examples, but rather encompasses all variations that are evident as a result of the teachings provided herein.

The HBV core protein modulators can be prepared in a number of ways. Schemes 1-9 illustrate the main routes employed for their preparation for the purpose of this application. To the chemist skilled in the art it will be apparent that there are other methodologies that will also achieve the preparation of these intermediates and Examples.

The following abbreviations are used:

A—DNA nucleobase adenine

ACN—acetonitrile

Ar—argon

BODIPY-FL—4,4-difluoro-5,7-dimethyl-4-bora-3a,4a-diaza-s-indacene-3-propionic acid (fluorescent dye)

Boc—tert-butoxycarbonyl

BnOH—benzyl alcohol

n-BuLi—n-butyl lithium

t-BuLi—t-butyl lithium

C—DNA nucleobase cytosine

CC₅₀—half-maximal cytotoxic concentration

CDI—1,1′-carbonyl diimidazole

CO₂—carbon dioxide

CuCN—copper (I) cyanide

DCE—dichloroethane

DCM—dichloromethane

Dess-Martin periodinane—1,1,1-triacetoxy-1,1-dihydro-1,2-benziodoxol-3(1H)-one

DIPEA—diisopropylethylamine

DIPE—di-isopropyl ether

DMAP—4-dimethylaminopyridine

DMF—N,N-dimethylformamide

DMP—Dess-Martin periodinane

DMSO—dimethyl sulfoxide

DNA—deoxyribonucleic acid

DPPA—diphenylphosphoryl azide

DTT—dithiothreitol

EC₅₀—half-maximal effective concentration

EDCI—N-(3-dimethylaminopropyl)-N′-ethylcarbodiimide hydrochloride

Et₂O—diethyl ether

EtOAc—ethyl acetate

EtOH—ethanol

FL-—five prime end labled with fluorescein

NEt₃—triethylamine

ELS—Evaporative Light Scattering

g—gram(s)

G—DNA nucleobase guanine

HBV—hepatitis B virus

HATU—2-(1H-7-azabenzotriazol-1-yl)-1,1,3,3-tetramethyl uronium hexafluorophosphate

HCl—hydrochloric acid

HEPES—4-(2-hydroxyethyl)-1-piperazineethanesulfonic acid

HOAt—1-hydroxy-7-azabenzotriazole

HOBt—1-hydroxybenzotriazole

HPLC—high performance liquid chromatography

IC₅₀—half-maximal inhibitory concentration

LC640-—3 prime end modification with fluorescent dye LightCycler® Red 640

LC/MS—liquid chromatography/mass spectrometry

LiAlH₄—lithium aluminium hydride

LiOH—lithium hydroxide

MeOH—methanol

MeCN—acetonitrile

MgSO₄—magnesium sulfate

mg—milligram(s)

min—minutes

mol—moles

mmol—millimole(s)

mL—millilitre(s)

MTBE—methyl tert-butyl ether

N₂—nitrogen

Na₂CO₃—sodium carbonate

NaHCO₃—sodium hydrogen carbonate

Na₂SO₄—sodium sulfate

NdeI —restriction enzyme recognizes CAATATG sites

NEt₃—triethylamine

NaH—sodium hydride

NaOH—sodium hydroxide

NH₃—ammonia

NH₄Cl—ammonium chloride

NMR—nuclear magnetic resonance

PAGE—polyacrylamide gel electrophoresis

PCR—polymerase chain reaction

qPCR—quantitative PCR

Pd/C—palladium on carbon

—PH—3 prime end phosphate modification

pTSA—4-toluene-sulfonic acid

Rt—retention time

r.t.—room temperature

sat.—saturated aqueous solution

SDS—sodium dodecyl sulfate

SI—selectivity index (═CC₅₀/EC₅₀)

STAB—sodium triacetoxyborohydride

T—DNA nucleobase thymine

TBAF—tetrabutylammonium fluoride

TFA—trifluoroacetic acid

THF—tetrahydrofuran

TLC—thin layer chromatography

Tris—tris(hydroxymethyl)-aminomethane

XhoI—restriction enzyme recognizes CATCGAG sites

In a preferred embodiment compounds of Formula I can be prepared as shown in General scheme 1 below.

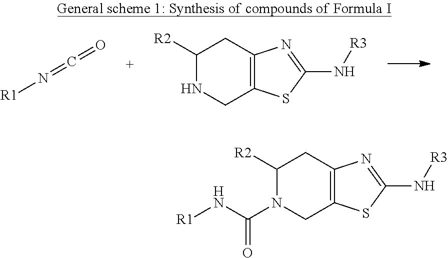

A coupling between an isocyanate and an appropriate amine (e.g. a suitably substituted 4H,5H,6H,7H-[1,3]thiazolo[5,4-c]pyridine) with methods known in literature (Pearson, A. J.; Roush, W. R.; Handbook of Reagents for Organic Synthesis, Activating Agents and Protecting Groups), e.g. with phenylisocyanate gives compounds of Formula I.

In a preferred embodiment compounds of Formula I can be prepared as shown in General scheme 2 below.

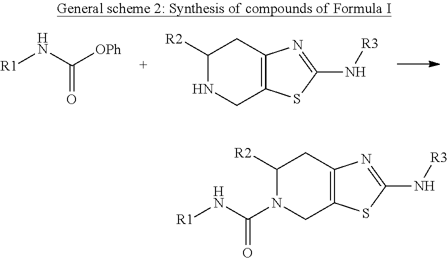

A coupling between a phenyl carbamate and an appropriate amine (e.g. a suitably substituted 4H,5H,6H,7H-[1,3]thiazolo[5,4-c]pyridine) with methods known in literature (Pearson, A. J.; Roush, W. R.; Handbook of Reagents for Organic Synthesis, Activating Agents and Protecting Groups), e.g. with a phenyl N-phenylcarbamate gives compounds of Formula I.

In a further embodiment the synthesis of compounds of Formula II follows General scheme 3.

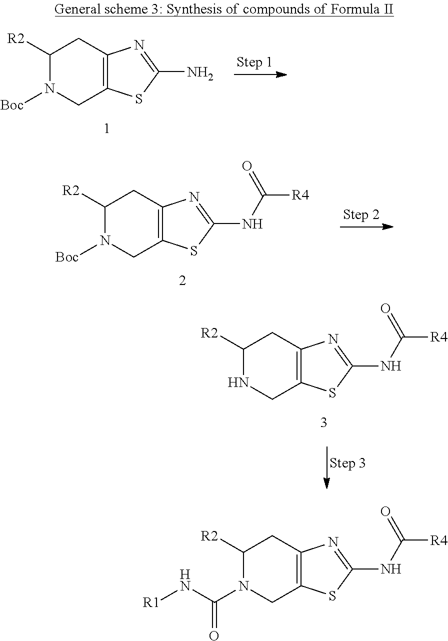

By methods known from the literature, in step 1 the compounds with general structure 1 described in general scheme 3 are acylated (P. N. Collier et al., J. Med. Chem., 2015, 58, 5684-5688), In step 2 deprotection of the nitrogen protective group (A. Isidro-Llobet et al., Chem. Rev., 2009, 109, 2455-2504), drawn as but not limited to Boc, e.g. with HCl gives an amine of general structure 3. A coupling in step 3 with methods known in literature (Pearson, A. J.; Roush, W. R.; Handbook of Reagents for Organic Synthesis, Activating Agents and Protecting Groups), e.g. with an isocyanate or activated carbamate results in compounds of Formula II.

In another embodiment an alternative synthesis of compounds of Formula II follows General scheme 4.

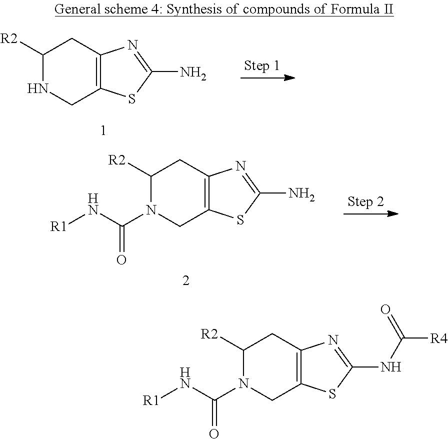

By methods known from the literature, in step 1 the compounds with general structure 1 described in general scheme 4 are derivatized (Pearson, A. J.; Roush, W. R.; Handbook of Reagents for Organic Synthesis, Activating Agents and Protecting Groups) e.g. with an isocyanate to give compounds of general structure 2. In step 2, acylation (P. N. Collier et al., J. Med. Chem., 2015, 58, 5684-5688) e,g, with an acid chloride gives compounds of Formula II.

In another embodiment an alternative synthesis of compounds of Formula III follows General scheme 5.

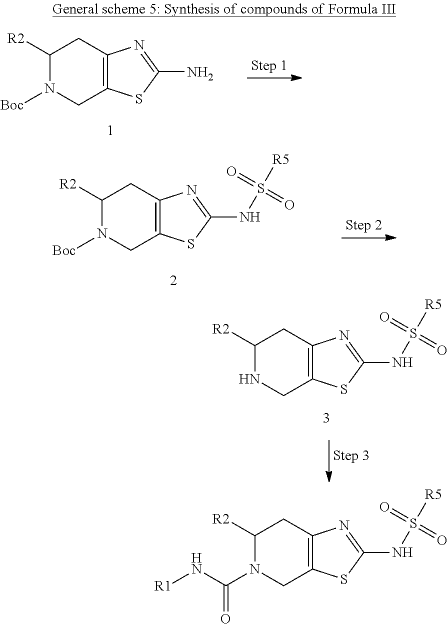

By methods known from the literature, in step 1 the compounds with general structure 1 described in general scheme 5 are sulfonylated (J. Inoue et al., Bioorg. Med. Chem., 2000, 8, 2167-2173), In step 2 deprotection of the nitrogen protective group (A. Isidro-Llobet et al., Chem. Rev., 2009, 109, 2455-2504), drawn as but not limited to Boc, e.g. with HCl gives an amine of general structure 3. A coupling in step 3 with methods known in literature (Pearson, A. J.; Roush, W. R.; Handbook of Reagents for Organic Synthesis, Activating Agents and Protecting Groups), e.g. with an isocyanate or activated carbamate results in compounds of Formula III.

In another embodiment an alternative synthesis of compounds of Formula III follows General scheme 6.

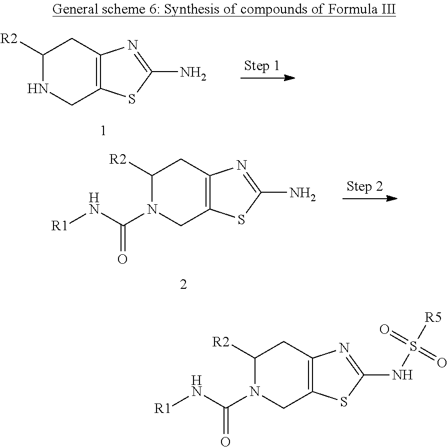

By methods known from the literature, in step 1 the compounds with general structure 1 described in general scheme 6 are coupled with methods known in the literature (Pearson, A. J.; Roush, W. R.; Handbook of Reagents for Organic Synthesis, Activating Agents and Protecting Groups) e.g. with an isocyanate to give compounds of general structure 2. In step 2, sulfonylation (J. Inoue et al., Bioorg. Med. Chem., 2000, 8, 2167-2173) e.g. with a sulfonyl chloride gives compounds of Formula III.

In another embodiment the synthesis of compounds of Formula IV follows General scheme 7.

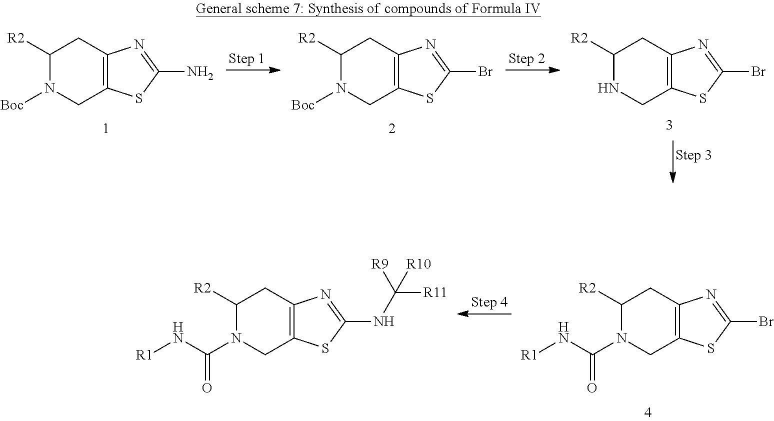

Compound 1 shown in General scheme 7 is converted into bromide 2 in a Sandmeyer reaction (X. Cao et al., J. Med. Chem., 2014, 57, 3687-3706). In step 2 deprotection of the nitrogen protective group (A. Isidro-Llobet et al., Chem. Rev., 2009, 109, 2455-2504), drawn as (but not limited to Boc) e.g. with TFA gives an amine of general structure 3. A coupling in step 3 with methods known in literature (Pearson, A. J.; Roush, W. R.; Handbook of Reagents for Organic Synthesis, Activating Agents and Protecting Groups), e.g. with a phenylisocyanate results in a compound with the general structure 4. By methods known from the literature, compounds with general structure 4 in step 4 are aminated (WO2014113191) to obtain compounds of Formula IV.

In another preferred embodiment the synthesis of compounds of Formula IV follows General scheme 8.

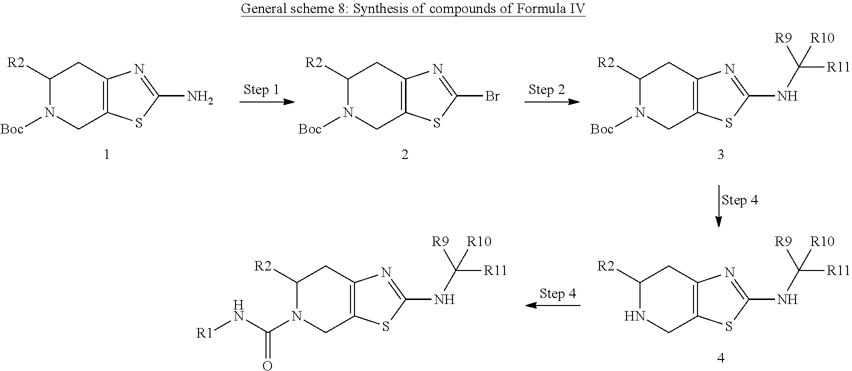

Compound 1 shown in General scheme 8 is in step 1 converted in a Sandmeyer reaction into bromide of general structure 2 (X. Cao et al., J. Med. Chem., 2014, 57, 3687-3706). Compound 2 described in general scheme 8 is in step 2 aminated (W02014113191), to obtain compounds with of general structure 3. In step 3 deprotection of the nitrogen protective group (A. Isidro-Llobet et al., Chem. Rev., 2009, 109, 2455-2504), drawn as but not limited to Boc, e.g. with HCl gives an amine of general structure 3. A coupling in step 4 with methods known in literature (Pearson, A. J.; Roush, W. R.; Handbook of Reagents for Organic Synthesis, Activating Agents and Protecting Groups), e.g. with a phenylisocyanate results in compounds of Formula IV.

In another preferred embodiment the synthesis of compounds of Formula IV follows General scheme 9.

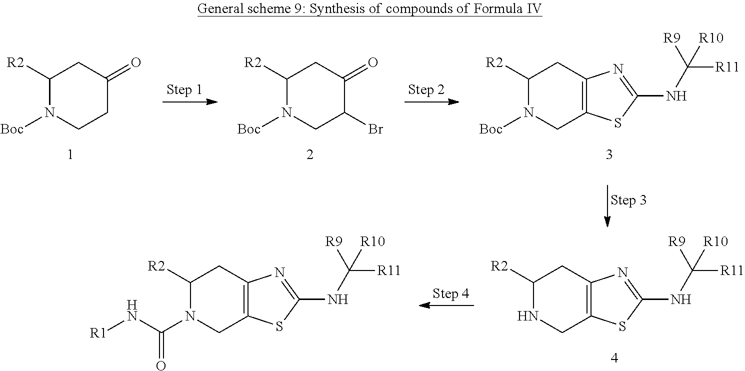

Ketone 1 shown in general scheme 9 is brominated to give the ct-bromo-ketone of general structure 2 (Provins et al., ChemMedChem 2012, 7(12) pp. 2087-2092). In step 2 condensation with a thiourea gives compounds of general structure 3. In step 3 deprotection of the nitrogen protective group (A. Isidro-Llobet et al., Chem. Rev., 2009, 109, 2455-2504), drawn as but not limited to Boc, e.g. with HCl gives an amine of general structure 4. A coupling reaction in step 4 with methods known in literature (Pearson, A. J.; Roush, W. R.; Handbook of Reagents for Organic Synthesis, Activating Agents and Protecting Groups), e.g. with a phenylisocyanate results in compounds a compound of Formula IV.

General procedure—Synthesis of Thioureas

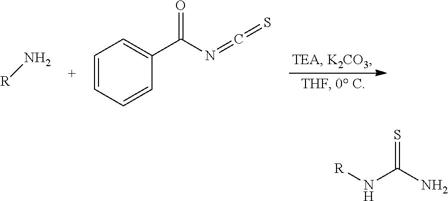

Triethylamine (7.66 mmol, 1.1 eq) was added to a solution of a corresponding amine hydrochloride (6.97 mmol, 1.0 eq) under an argon atmosphere in dry THF (10 mL) at 0° C. (ice bath). The resulting mixture was stirred for 10 min followed by the addition of benzoyl isothiocyanate (7.66 mmol, 1.1 eq). After removing the ice bath, the reaction mixture was allowed to warm to RT and stirred overnight. After the completion of reaction, the solution was concentrated under reduced pressure and the residue was re-suspended in a mixture of water (5 mL) and methanol (5 mL). Potassium carbonate (15.33 mmol, 2.2 eq) was added to the resulting suspension. The mixture was stirred overnight at RT and concentrated under reduced pressure (co-evaporation with ethyl acetate). The obtained solid was re-suspended in 1:1 DCM/MeOH (150 mL) and filtered off. The filtrate was concentrated under reduced pressure to afford a crude thiourea which was further purified by RP-HPLC.

The following thioureas were prepared as described above.

### 1-(3,3-Difluorocyclobutypthiourea

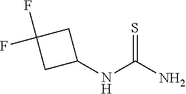

Yield 725.0 mg (62.6%).

¹H NMR (500 MHz, DMSO-d6) δ (ppm) 2.48 (m, 2H), 2.90 (m, 2H), 4.37 (m, 1H), 6.93 (m, 1H), 7.44 (m, 1H), 7.97 (m, 1H).

LCMS(ESI): [M+H]+ m/z: calc. 167.0; found 167.2; Rt=0.72 min

### 1-((1r,3r)-3-Fluorocyclobutyl)thiourea

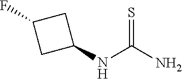

Yield 120.7 mg (16.8%).

¹H NMR (500 MHz, DMSO-d6) δ (ppm) 2.28 (m, 2H), 2.43 (m, 2H), 4.61 (m, 1H), 5.16 (m, 1H), 6.96 (m, 1H), 7.52 (m, 1H), 8.03 (m, 1H).

LCMS(ESI): [M+H]+ m/z: calc. 149.0; found 149.0; Rt=0.49 min

### 1-(2,2-Difluorocyclobutyl)thiourea

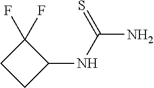

Yield 50.4 mg (41.4%).

¹H NMR (400 MHz, DMSO-d6) δ (ppm) 1.56 (m, 1H), 2.15 (m, 1H), 2.30 (m, 2H), 5.18 (m, 1H), 7.23 (m, 2H), 8.02 (m, 1H).

LCMS(ESI): [M+H]+ m/z: calc. 167.0; found 166.9; Rt=0.71 min

### 1-(3,3-Difluoro-1-methylcyclobutypthiourea

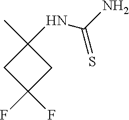

Yield 415.0 mg (72%).

¹H NMR (500 MHz, DMSO-d6) δ (ppm) 1.59 (s, 3H), 2.63 (m, 2H), 2.90 (q, 2H), 6.78 (m, 2H), 7.93 (s, 1H).

LCMS(ESI): [M+H]+ m/z: calc. 181.0; found 181.2; Rt=0.87 min

### 1-(3,3-Difluoro-1-(hydroxymethypcyclobutypthiourea

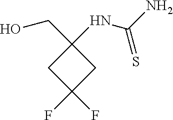 [SMILES: NC(=S)NC1(CO)CC(F)(F)C1]

Yield 184.0 mg (36.2%).

¹H NMR (400 MHz, DMSO-d6) δ (ppm) 2.75 (m, 4H), 3.68 (m, 2H), 5.22 (m, 1H), 6.96 (m, 2H), 7.91 (s, 1H).

LCMS(ESI): [M+H]+ m/z: calc. 197.0; found 197.2; Rt=0.79 min

### 1-(3,3-Difluoro-1-(methoxymethyl)cyclobutypthiourea

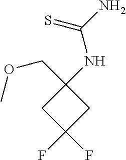

Yield 325.0 mg (38.7%).

¹H NMR (500 MHz, DMSO-d6) δ (ppm) 2.78 (m, 4H), 3.34 (m, 3H), 3.75 (m, 2H), 6.90 (m, 2H), 7.95 (m, 1H).

LCMS(ESI): [M+H]+ m/z: calc. 211.0; found 211.0; Rt=0.90 min

### 1-(1-(Trifluoromethyl)cyclobutypthiourea

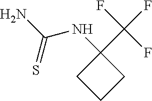 [SMILES: NC(=S)NC1(C(F)(F)F)CCC1]

Yield 94.0 mg (83.2%).

¹H NMR (500 MHz, DMSO-d6) δ (ppm) 1.89 (m, 2H), 2.44 (m, 2H), 2.52 (m, 2H), 8.19 (m, 3H).

LCMS(ESI): [M+H]+ m/z: calc. 199.0; found 199.0; Rt=0.69 min

### 1-(1-(Methoxymethyl)cyclobutyl)thiourea

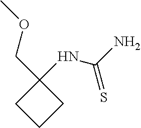

Yield 515.0 mg (44.8%).

¹H NMR (500 MHz, DMSO-d6) δ (ppm) 1.79 (m, 2H), 2.16 (m, 4H), 3.35 (s, 3H), 3.77 (m, 2H), 6.59 (m, 2H), 7.55 (m, 1H).

LCMS(ESI): [M+H]+ m/z: calc. 175.0; found 175.2; Rt=0.79 min

### 1-(1-(Methoxymethyl)cyclopropyl)thiourea

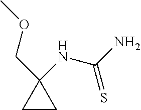

Yield 1.11 g (94.9%).

¹H NMR (500 MHz, DMSO-d6) δ (ppm) 0.79 (m, 4H), 3.11 (s, 3H), 3.31 (m, 2H), 6.80 (m, 1H), 7.50 (m, 1H), 7.86 (m, 1H).

LCMS(ESI): [M+H]+ m/z: calc. 161.1; found 161.1; Rt=0.62 min

### 1-(1-(Trifluoromethypcyclopropypthiourea

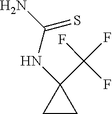 [SMILES: NC(=S)NC1(C(F)(F)F)CC1]

Yield 405.0 mg (35.5%).

¹H NMR (400 MHz, DMSO-d6) δ (ppm) 1.11 (m, 2H), 1.26 (m, 2H), 7.13 (m, 1H), 7.94 (m, 1H), 8.39 (m, 1H).

LCMS(ESI): [M+H]+ m/z: calc. 185.0; found 185.2; Rt=0.63 min

### 1((3,3-Difluoro-1-hydroxycyclobutyl)methypthiourea

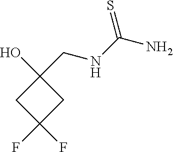 [SMILES: NC(=S)NCC1(O)CC(F)(F)C1]

Yield 35.7%.

¹H NMR (500 MHz, DMSO-d6) δ (ppm) 2.42 (m, 2H), 2.74 (m, 2H), 3.60 (m, 2H), 7.26 (m, 2H), 7.76 (m, 2H).

LCMS(ESI): [M+H]+ m/z: calc. 197.0; found 197.0; Rt=0.69 min

### 1((3,3-Difluorocyclobutypmethypthiourea

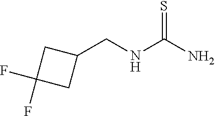 [SMILES: NC(=S)NCC1CC(F)(F)C1]

Yield 169.1 mg (24.7%).

¹H NMR (500 MHz, CDCl3) δ (ppm) 2.29 (m, 2H), 2.48 (m, 1H), 2.74 (m, 2H), 3.56 (m, 2H), 5.80 (m, 2H), 6.26 (m, 1H).

LCMS(ESI): [M+H]+ m/z: calc. 181.0; found 181.0; Rt=0.81 min

### N-Methyl-1-(thioureidomethyl)cyclobutanecarboxamide

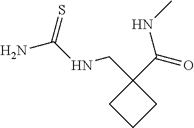

Yield 79.2 mg (17.6%).

¹H NMR (400 MHz, DMSO-d6) δ (ppm) 1.70 (m, 2H), 1.88 (m, 2H), 2.17 (m, 2H), 2.61 (s, 3H), 3.75 (m, 2H), 7.09 (m, 2H), 7.29 (m, 1H), 7.67 (m, 1H).

LCMS(ESI): [M+H]+ m/z: calc. 202.1; found 202.2; Rt=0.68 min

### 1-((1-Methoxycyclobutyl)methyl)thiourea

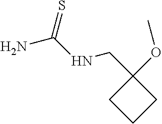

Yield 97.0 mg (64.1%).

¹H NMR (400 MHz, DMSO-d6) δ (ppm) 1.56 (m, 1H), 1.62 (m, 1H), 1.82 (m, 2H), 2.02 (m, 2H), 3.09 (s, 3H), 3.66 (d, 2H), 7.05 (m, 2H), 7.39 (m, 1H).

LCMS(ESI): [M+H]+ m/z: calc. 175.1; found 175.2; Rt=0.87 min

### 1-(Bicyclo[1.1.1]pentan-1-yl)thiourea

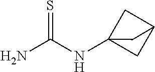

Yield 192.5 mg, (40.4%).

¹H NMR (400 MHz, DMSO-d6) δ (ppm) 2.05 (s, 6H), 2.38 (m, 1H), 6.76 (m, 2H), 8.25 (m, 1H).

LCMS(ESI): [M+H]+ m/z: calc. 143.0; found 143.0; Rt=0.77 min

### 1-((1s,3s)-3-Hydroxy-3-methylcyclobutyl)thiourea

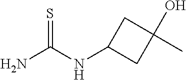

Yield 190.0 mg (32.6%).

¹H NMR (500 MHz, DMSO-d6) δ (ppm) 1.89 (m, 4H), 2.27 (m, 3H), 4.04 (m, 1H), 4.92 (m, 1H), 6.84 (m, 2H), 7.80 (m, 1H).

LCMS(ESI): [M+H]+ m/z: calc. 161.0; found 161.1; Rt=0.49 min

### 1-((1r,3r)-3-Methoxycyclobutyl)thiourea

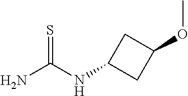

Yield 57.8 mg (49.8%).

¹H NMR (400 MHz, DMSO-d6) δ (ppm) 2.18 (m, 4H), 3.11 (s, 3H), 3.89 (m, 1H), 4.48 (m, 1H), 6.88 (m, 1H), 7.37 (m, 1H), 7.92 (m, 1H).

LCMS(ESI): [M+H]+ m/z: calc. 161.0; found 161.2; Rt=0.62 min

### 1-((1s,3s)-3-Methoxycyclobutyl)thiourea

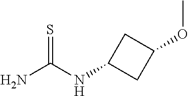

Yield 57.8 mg (49.8%).

¹H NMR (500 MHz, DMSO-d6) δ (ppm) 2.58 (m, 2H), 3.10 (s, 3H), 3.54 (m, 1H), 4.10 (m, 1H), 6.87 (m, 1H), 7.39 (m, 1H), 7.90 (m, 1H).

LCMS(ESI): [M+H]+ m/z: calc. 161.0; found 161.0; Rt=0.68 min

### 1-(3-(Difluoromethoxy)cyclobutyl)thiourea

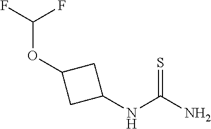 [SMILES: NC(=S)NC1CC(OC(F)F)C1]

Yield 121.0 mg (14.4%).

¹H NMR (500 MHz, DMSO-d6) δ (ppm) 2.06 (m, 2H), 2.26 (m, 1H), 2.68 (m, 2H), 4.32 (m, 2H), 6.61 (m, 1H), 7.96 (m, 2H).

LCMS(ESI): [M+H]+ m/z: calc. 197.0; found 197.0; Rt=0.81 min

### 1-(3-Cyanobicyclo[1.1.1]pentan-1-yl)thiourea

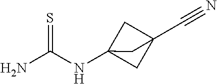 [SMILES: N#CC12CC(NC(N)=S)(C1)C2]

Yield 78.4 mg (11.2%).

¹H NMR (400 MHz, DMSO-d6) δ (ppm) 2.56 (m, 6H), 7.20 (m, 2H), 8.46 (s, 1H).

LCMS(ESI): [M+H]+ m/z: calc. 168.0; found 168.0; Rt=0.75 min

### 1-(2-Cyclopropyl-2,2-difluoroethyl)thiourea

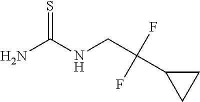 [SMILES: NC(=S)NCC(F)(F)C1CC1]

Yield 110.0 mg (20.5%).

¹H NMR (400 MHz, CDCl3) δ (ppm) 0.64 (m, 4H), 1.27 (m, 1H), 4.04 (m, 2H), 6.16 (m, 2H), 6.83 (m, 1H).

LCMS(ESI): [M+H]+ m/z: calc. 181.0; found 181.0; Rt=0.90 min

Compound Identification—NMR

For a number of compounds, NMR spectra were recorded using a Bruker DPX400 spectrometer equipped with a 5 mm reverse triple-resonance probe head operating at 400 MHz for the proton and 100 MHz for carbon. Deuterated solvents were chloroform-d (deuterated chloroform, CDCl₃) or d6-DMSO (deuterated DMSO, d6-dimethylsulfoxide). Chemical shifts are reported in parts per million (ppm) relative to tetramethylsilane (TMS) which was used as internal standard.

Compound Identification—HPLC/MS

For a number of compounds, LC-MS spectra were recorded using the following analytical methods.

Method A

Column—Reverse phase Waters Xselect CSH C18 (50×2.1 mm, 3.5 micron)

Flow—0.8 mL/min, 25 degrees Celsius

Eluent A—95% acetonitrile+5% 10 mM ammonium carbonate in water (pH 9)

Eluent B—10 mM ammonium carbonate in water (pH 9)

Linear gradient t=0 min 5% A, t=3.5 min 98% A. t=6 min 98% A

Method A2

Column—Reverse phase Waters Xselect CSH C18 (50×2.1 mm, 3.5 micron)

Flow—0.8 mL/min, 25 degrees Celsius

Eluent A—95% acetonitrile+5% 10 mM ammonium carbonate in water (pH 9)

Eluent B—10 mM ammonium carbonate in water (pH 9)

Linear gradient t=0 min 5% A, t=4.5 min 98% A. t=6 min 98% A

Method B

Column—Reverse phase Waters Xselect CSH C18 (50×2.1 mm, 3.5 micron)

Flow—0.8 mL/min, 35 degrees Celsius

Eluent A—0.1% formic acid in acetonitrile

Eluent B—0.1% formic acid in water

Linear gradient t=0 min 5% A, t=3.5 min 98% A. t=6 min 98% A

Method B2

Column—Reverse phase Waters Xselect CSH C18 (50×2.1 mm, 3.5 micron)

Flow—0.8 mL/min, 40 degrees Celsius

Eluent A—0.1% formic acid in acetonitrile

Eluent B—0.1% formic acid in water

Linear gradient t=0 min 5% A, t=4.5 min 98% A. t=6 min 98% A

Method C

Column—Reverse phase Waters Xselect CSH C18 (50×2.1 mm, 3.5 micron)

Flow—1 mL/min, 35 degrees Celsius

Eluent A—0.1% formic acid in acetonitrile

Eluent B—0.1% formic acid in water

Linear gradient t=0 min 5% A, t=1.6 min 98% A. t=3 min 98% A

Method D

Column—Phenomenex Gemini NX C18 (50×2.0 mm, 3.0 micron)

Flow—0.8 mL/min, 35 degrees Celsius

Eluent A—95% acetonitrile+5% 10 mM ammoniumbicarbonate in water

Eluent B—10 mM ammoniumbicarbonate in water pH=9.0

Linear gradient t=0 min 5% A, t=3.5 min 98% A. t=6 min 98% A

Method E

Column—Phenomenex Gemini NX C18 (50×2.0 mm, 3.0 micron)

Flow—0.8 mL/min, 25 degrees Celsius

Eluent A—95% acetonitrile+5% 10 mM ammoniumbicarbonate in water

Eluent B—10 mM ammonium bicarbonate in water (pH 9)

Linear gradient t=0 min 5% A, t=3.5 min 30% A. t=7 min 98% A, t=10 min 98% A

Method F

Column—Waters XSelect HSS C18 (150×4.6 mm, 3.5 micron)

Flow—1.0 mL/min, 25 degrees Celsius

Eluent A—0.1% TFA in acetonitrile

Eluent B—0.1% TFA in water

Linear gradient t=0 min 2% A, t=1 min 2% A, t=15 min 60% A, t=20 min 60% A

Method G

Column—Zorbax SB-C18 1.8 μm 4.6×15 mm Rapid Resolution cartridge (PN 821975-932)

Flow—3 mL/min

Eluent A—0.1% formic acid in acetonitrile

Eluent B—0.1% formic acid in water

Linear gradient t=0 min 0% A, t=1.8 min 100% A

Method H

Column—Waters Xselect CSH C18 (50×2.1 mm, 2.5 micron)

Flow—0.6 mL/min

Eluent A—0.1% formic acid in acetonitrile

Eluent B—0.1% formic acid in water

Linear gradient t=0 min 5% A, t=2.0 min 98% A, t=2.7 min 98% A

Method J

Column—Reverse phase Waters Xselect CSH C18 (50×2.1 mm, 2.5 micron)

Flow—0.6 mL/min

Eluent A—100% acetonitrile

Eluent B—10 mM ammonium bicarbonate in water (pH 7.9)

Linear gradient t=0 min 5% A, t=2.0 min 98% A, t=2.7 min 98% A

The following examples illustrate the preparation and properties of some specific compounds of the invention.

### Example 1

### 2-amino -N-(3-chloro -4-fluorophenyl)-4H,5H,6H,7H-[1,3] thiazolo[5,4-c]pyridine-5-carboxamide

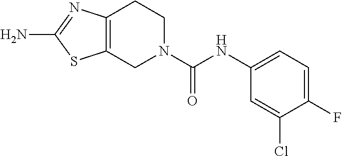 [SMILES: Nc1nc2c(s1)CN(C(=O)Nc1ccc(F)c(Cl)c1)CC2]

Rt (Method A) 2.95 mins, m/z 327/329 [M+H]+

¹H NMR (400 MHz, DMSO-d6) δ 8.79 (s, 1H), 7.75 (dd, J=6.9, 2.6 Hz, 1H), 7.42 (ddd, J=9.1, 4.4, 2.7 Hz, 1H), 7.29 (t, J=9.1 Hz, 1H), 6.82 (s, 2H), 4.47-4.40 (m, 2H), 3.75-3.67 (m, 2H), 2.56-2.51 (m, 2H).

### Example 2

### N-(3-chloro-4-fluorophenyl)-2-{[(1r,3r)-3-hydroxycyclobutyl]amino}-4H,5H,6H,7H-[1,3]thiazolo[5,4-c]pyridine-5-carboxamide

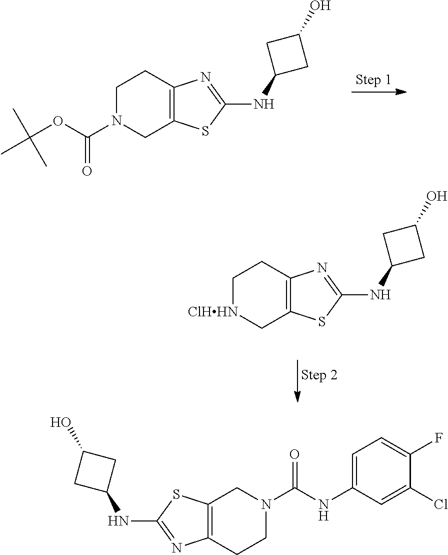

Step 1: To tert-butyl 2-(((1r,3r)-3-hydroxycyclobutyl)amino)-6,7-dihydrothiazolo[5,4-c]pyridine-5(4H)-carboxylate (1.77 g, 5.44 mmol) was added 4M HCl in dioxane (15 mL, 60 mmol). The mixture was stirred at room temperature for 4 hours, then concentrated in vacuo. The residue was stripped with toluene (twice) and CH₂Cl₂ to obtain 1.54 grams of a white solid that was used without further purification.

Step 2: To a solution of (1r,3r)-3-((4,5,6,7-tetrahydrothiazolo[5,4-c]pyridin-2-yl)amino)cyclobutan-1-ol hydrochloride (50 mg, 0.191 mmol) and DIPEA (0.167 mL, 0.955 mmol) in dry N,N-dimethylformamide (2 mL) was added 2-chloro-1-fluoro-4-isocyanatobenzene (0.024 mL, 0.191 mmol). The mixture was stirred at r.t. for 30 minutes then water was added. The product was extracted with EtOAc (2×4 mL), and the combined organic extracts were washed with brine (3×10 mL), dried over Na₂SO₄, filtered and concentrated in vacuo to give a brown oil. Trituration with Et₂O gave an off-white solid that was purified by HPLC to give N-(3-chloro-4-fluorophenyl)-2-{[(1r,3r)-3-hydroxycyclobutyl]amino}-4H,5H,6H,7H-[1,3]thiazolo[5,4-c]pyridine-5-carboxamide as a white solid (12 mg, 16% yield).

Rt (Method B) 2.37 mins, m/z 397/399 [M+H]+

¹H NMR (400 MHz, DMSO-d6) δ 8.80 (s, 1H), 7.79-7.69 (m, 2H), 7.42 (ddd, J=9.1, 4.4, 2.6 Hz, 1H), 7.29 (t, J=9.1 Hz, 1H), 5.04 (m, 1H), 4.45 (d, J=1.9 Hz, 2H), 4.28 (q, J=6.0 Hz, 1H), 4.00 (q, J=6.1 Hz, 1H), 3.71 (t, J=5.7 Hz, 2H), 2.55 (t, J=3.8 Hz, 2H), 2.14 (t, J=6.1 Hz, 4H).

### Example 3

N-(3-chloro-4-fluorophenyl)-2-{[1-(hydroxymethyl)cyclobutyl]amino}-4H,5H,6H,7H-[1,3]thiazolo[5,4-c]pyridine-5-carboxamide

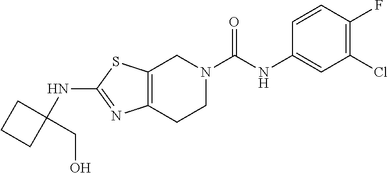 [SMILES: O=C(Nc1ccc(F)c(Cl)c1)N1CCc2nc(NC3(CO)CCC3)sc2C1]

Rt (Method A) 3.15 mins, m/z 411/413 [M+H]+

¹H NMR (400 MHz, DMSO-d6) δ 8.81 (s, 1H), 7.75 (dd, J=6.9, 2.6 Hz, 1H), 7.57 (s, 1H), 7.45-7.39 (m, 1H), 7.29 (t, J=9.1 Hz, 1H), 4.96 (t, J=5.6 Hz, 1H), 4.46-4.41 (m, 2H), 3.75-3.67 (m, 2H), 3.62 (d, J=5.6 Hz, 2H), 2.55-2.51 (m, 2H), 2.14-2.04 (m, 4H), 1.89-1.75 (m, 1H), 1.75-1.65 (m, 1H).

### Example 4

### N-(3-chloro-4-fluorophenyl)-2-[(oxolan-3-yl)amino]-4H,5H,6H,7H-[1,3]thiazolo[5,4-c]pyridine-5-carboxamide

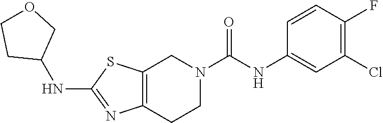 [SMILES: O=C(Nc1ccc(F)c(Cl)c1)N1CCc2nc(NC3CCOC3)sc2C1]

Rt (Method A) 3.01 mins, m/z 397/399 [M+H]+

¹H NMR (400 MHz, DMSO-d6) δ 8.81 (s, 1H), 7.75 (dd, J=6.9, 2.6 Hz, 1H), 7.70 (d, J=6.2 Hz, 1H), 7.46-7.38 (m, 1H), 7.29 (t, J=9.1 Hz, 1H), 4.48-4.42 (m, 2H), 4.19 (s, 1H), 3.84-3.75 (m, 2H), 3.75-3.64 (m, 3H), 3.56 (dd, J=9.0, 3.4 Hz, 1H), 2.60-2.53 (m, 2H), 2.20-2.05 (m, 1H), 1.86-1.74 (m, 1H).

### Example 5

### N-(3-chloro-4-fluorophenyl)-2-{[(oxolan-3-yl)methyl]amino}-4H,5H,6H,7H-[1,3]thiazolo [5,4-c]pyridine-5-carboxamide

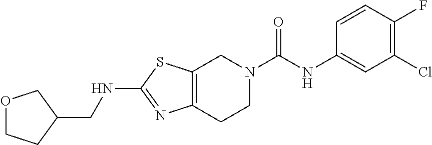 [SMILES: O=C(Nc1ccc(F)c(Cl)c1)N1CCc2nc(NCC3CCOC3)sc2C1]

Rt (Method A) 3.06 mins, m/z 411/413 [M+H]+

¹H NMR (400 MHz, DMSO-d6) δ 8.81 (s, 1H), 7.75 (dd, J=7.0, 2.7 Hz, 1H), 7.63-7.57 (m, 1H), 7.45-7.38 (m, 1H), 7.33-7.25 (m, 1H), 4.47-4.41 (m, 2H), 3.76-3.65 (m, 4H), 3.65-3.56 (m, 1H), 3.42 (dd, J=8.6, 5.4 Hz, 1H), 3.19-3.12 (m, 2H), 2.59-2.52 (m, 2H), 2.46-2.43 (m, 1H), 1.98-1.90 (m, 1H), 1.61-1.49 (m, 1H).

### Example 6

### N-(3-chloro-4-fluorophenyl)-2-{[(1-hydroxycyclobutyl)methyl]amino}-4H,5H,6H,7H-[1,3]thiazolo[5,4-c]pyridine-5-carboxamide

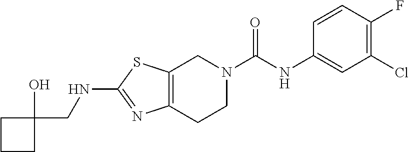 [SMILES: O=C(Nc1ccc(F)c(Cl)c1)N1CCc2nc(NCC3(O)CCC3)sc2C1]

Rt (Method B) 2.51 mins, m/z 411/413 [M+H]+

¹H NMR (400 MHz, DMSO-d6) δ 8.79 (s, 1H), 7.75 (dd, J=6.9, 2.6 Hz, 1H), 7.45-7.37 (m, 2H), 7.29 (t, J=9.1 Hz, 1H), 5.27 (s, 1H), 4.43 (t, J=2.0 Hz, 2H), 3.72 (t, J=5.7 Hz, 2H), 2.56-2.51 (m, 2H), 2.05-1.85 (m, 4H), 1.68-1.57 (m, 1H), 1.52-1.39 (m, 1H).

### Example 7

N-(3-chloro-4-fluorophenyl)-2-{[(1s,4s)-4-hydroxycyclohexyl]amino}-4H,5H,6H,7H-[1,3]thiazolo[5,4-c]pyridine-5-carboxamide

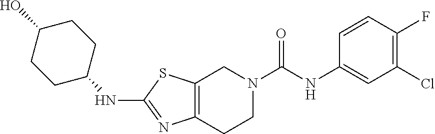 [SMILES: O=C(Nc1ccc(F)c(Cl)c1)N1CCc2nc(N[C@H]3CC[C@@H](O)CC3)sc2C1]

Rt (Method B) 2.44 mins, m/z 425/427 [M+H]+

¹H NMR (400 MHz, DMSO-d6) δ 8.81 (s, 1H), 7.75 (dd, J=6.9, 2.6 Hz, 1H), 7.51-7.38 (m, 2H), 7.30 (t, J=9.1 Hz, 1H), 4.42 (m, 3H), 3.78-3.43 (m, 4H), 2.53-2.51 (m, 2H), 1.81-1.33 (m, 8H).

### Example 8

### N-{5-[(3-chloro-4-fluorophenyl)carbamoyl]-4H,5H,6H,7H-[1,3]thiazolo[5,4-c]pyridin-2-yl}-1-methylpiperidine-4-carboxamide formate

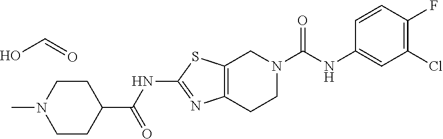

Rt (Method B) 2.39 mins, m/z 452/454 [M+H]+

¹H NMR (400 MHz, DMSO-d6) δ 12.00 (s, 1H), 8.86 (s, 1H), 8.21 (s, 1H), 7.75 (m, 1H), 7.43 (m, 1H), 7.30 (t, J=9.1 Hz, 1H), 4.61 (m, 2H), 3.78 (t, J=5.7 Hz, 2H), 2.82 (m, 2H), 2.70 (t, J=5.9 Hz, 2H), 2.41 (m, 1H), 2.18 (s, 3H), 1.90 (m, 2H), 1.82-1.70 (m, 2H), 1.63 (m, 2H).

### Example 9

### N-(3-chloro-4-fluorophenyl)-2-{[(3,3-difluoro-1-hydroxycyclobutyl)methyl]amino}-4H,5H,6H,7H-[1,3]thiazolo[5,4-c]pyridine-5-carboxamide

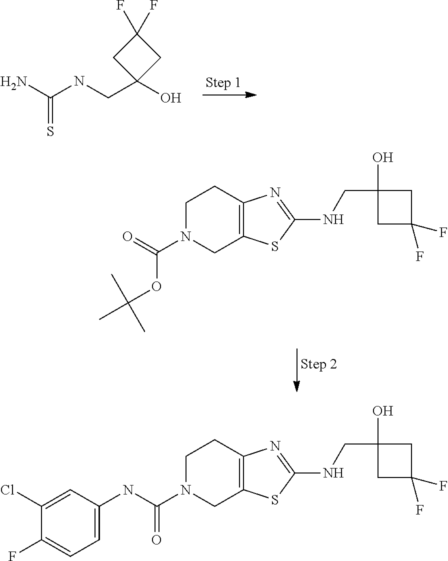

Step 1: A solution of 1-((3,3-difluoro-1-hydroxycyclobutyl)methyl)thiourea (0.050 g, 0.255 mmol) in ethanol (2 mL) was added to tert-butyl 3-bromo-4-oxopiperidine-1-carboxylate (0.071 g, 0.255 mmol) and sodium bicarbonate (0.032 g, 0.382 mmol). The mixture was stirred at 75° C. The mixture was cooled to r.t. and concentrated. The residue was partitioned between water and dichloromethane. The organic layer was collected by phase separator and concentrated to give tert-butyl 2-{[(3,3-difluoro-1-hydroxycyclobutyl)methyl]amino}-4H,5H,6H,7H-[1,3]thiazolo[5,4-c]pyridine-5-carboxylate as a white solid (104 mg, 100% yield).

Step 2: Tert-butyl 2-(((3,3-difluoro-1-hydroxycyclobutyl)methyl)amino)-6,7-dihydrothiazolo[5,4-c]pyridine-5(4H)-carboxylate (0.104 g, 0.277 mmol) was dissolved in 4M HCl in dioxane (2 mL, 8.00 mmol) and stirred for lh. The mixture was then concentrated, and the residue dissolved in dry N,N-dimethylformamide (1 mL). Triethylamine (0.154 mL, 1.108 mmol) was added, followed by 2-chloro-1-fluoro-4-isocyanatobenzene (0.035 ml, 0.277 mmol). The mixture was filtered and purified by HPLC to give N-(3-chloro-4-fluorophenyl)-2-{[(3,3-difluoro-1-hydroxycyclobutyl)methyl]amino}-4H,5H,6H,7H-[1,3]thiazolo[5,4-c]pyridine-5-carboxamide as an off-white solid (49 mg, 40% yield).

Rt (Method H) 1.1 mins, m/z 446/448 [M+H]+

¹H NMR (400 MHz, DMSO-d6) δ 8.81 (s, 1H), 7.75 (dd, J=6.9, 2.6 Hz, 1H), 7.62 (t, J=5.9 Hz, 1H), 7.42 (ddd, J=9.1, 4.3, 2.7 Hz, 1H), 7.29 (t, J=9.1 Hz, 1H), 5.80 (s, 1H), 4.48-4.40 (m, 2H), 3.76-3.68 (m, 2H), 3.45-3.38 (m, 2H), 2.83-2.69 (m, 2H), 2.58-2.47 (m, 4H).

### Example 10

### N-(3-chloro-4-fluorophenyl)-2-(oxolane-3-amido)-4H,5H,6H,7H-[1,3]thiazolo[5,4-c]pyridine-5-carboxamide

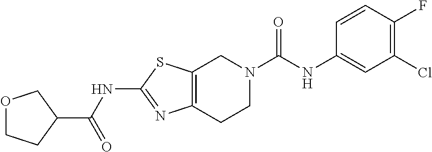 [SMILES: O=C(Nc1nc2c(s1)CN(C(=O)Nc1ccc(F)c(Cl)c1)CC2)C1CCOC1]

Rt (Method A) 2.98 mins, m/z 425/427 [M+H]+

¹H NMR (400 MHz, DMSO-d6) δ 12.14 (s, 1H), 8.85 (s, 1H), 7.75 (dd, J=6.9, 2.6 Hz, 1H), 7.58-7.36 (m, 1H), 7.30 (t, J=9.1 Hz, 1H), 4.62 (s, 2H), 3.91 (t, J=8.2 Hz, 1H), 3.87-3.59 (m, 5H), 3.29-3.15 (m, 1H), 2.78-2.60 (m, 2H), 2.23-1.96 (m, 2H).

### Example 11

### N-(3-chloro-4-fluorophenyl)-2-(oxane-4-amido)-4H,5H,6H,7H-[1,3]thiazolo[5,4-c]pyridine-5-carboxamide

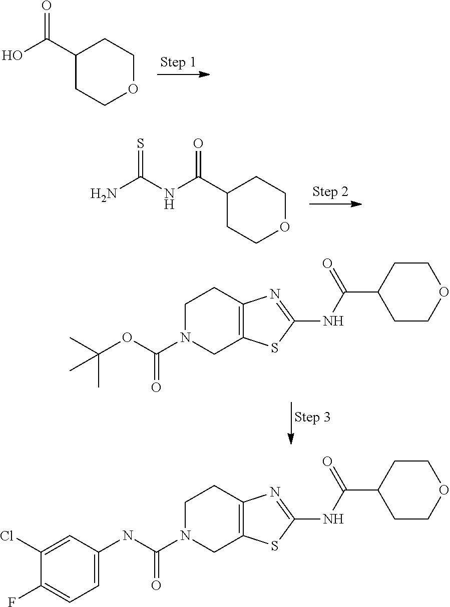

Step 1: A mixture of tetrahydro-2H-pyran-4-carboxylic acid (100 mg, 0.768 mmol) in thionyl chloride (2 mL, 27.4 mmol) was heated at reflux (75° C.) for 2 hours. The mixture was then concentrated and the residue re-dissolved in toluene (1 mL). Thiourea (292 mg,3.84 mmol) was added and the mixture heated (110° C.) for 2 hours. The mixture was cooled and stirred at r.t. overnight, then concentrated. Product was extracted with EtOAc, and the combined organic layers were washed with brine, dried over Na₂SO₄, filtered and concentrated in vacuo to give (oxane-4-carbonyl)thiourea as a yellow solid (88 mg, 28% yield).

Step 2: To a mixture of tert-butyl 3-bromo-4-oxopiperidine-1-carboxylate (191 mg, 0.685 mmol) and (oxane-4-carbonyl)thiourea (129 mg, 0.685 mmol) in ethanol (5 mL) was added sodium bicarbonate (86 mg, 1.028 mmol). The mixture was stirred at 80° C. overnight, cooled and concentrated in vacuo. CH₂Cl₂ was added, the solids were removed by filtration and rinsed with CH₂Cl₂. The filtrate was concentrated in vacuo and purified using flash chromatography (30-100% EtOAc in heptane) to give a tert-butyl 2-(oxane-4-amido)-4H,5H,6H,7H-[1,3]thiazolo[5,4-c]pyridine-5-carboxylate as a yellow foam (103 mg, 41% yield).

Step 3: A mixture of tert-butyl 2-(tetrahydro-2H-pyran-4-carboxamido)-6,7-dihydrothiazolo[5,4-c]pyridine-5(4H)-carboxylate (103 mg, 0.280 mmol) and 4M HCl in dioxane (2 ml, 8.00 mmol) was stirred at rt for 2 hours (s1). The mixture was concentrated in vacuo and stripped with toluene (twice) and EtOAc. To the residue (N-(4,5,6,7-tetrahydrothiazolo[5,4-c]pyridin-2-yl)tetrahydro-2H-pyran-4-carboxamide hydrochloride, 88 mg, 0.290 mmol) was added dry N,N-dimethylformamide (1 mL), 2-chloro-1-fluoro-4-isocyanatobenzene (0.036 mL, 0.290 mmol) and triethylamine (0.202 mL, 1.448 mmol). The mixture was stirred at r.t. overnight. A few drops of water were added, and the resulting solution was purified by reverse phase chromatography to give N-(3-chloro-4-fluorophenyl)-2-(oxane-4-amido)-4H,5H,6H,7H-[1,3]thiazolo[5,4-c]pyridine-5-carboxamide as a white solid (33 mg, 25% yield).

Rt (Method B) 3.12 mins, m/z 439/441 [M+H]+

¹H NMR (400 MHz, DMSO-d6) δ 12.02 (s, 1H), 8.86 (s, 1H), 7.75 (dd, J=6.9, 2.6 Hz, 1H), 7.48-7.38 (m, 1H), 7.30 (t, J=9.1 Hz, 1H), 4.62 (s, 2H), 3.96-3.84 (m, 2H), 3.78 (t, J=5.7 Hz, 2H), 3.41-3.34 (m, 1H), 3.31-3.19 (m, 1H), 2.82-2.64 (m, 3H), 1.79-1.53 (m, 4H).

### Example 12

### N-(3-chloro-4-fluorophenyl)-2-{[(1-hydroxycyclopropyl)methyl]amino}-4H,5H,6H,7H-[1,3]thiazolo[5,4-c]pyridine-5-carboxamide

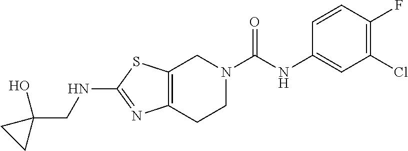 [SMILES: O=C(Nc1ccc(F)c(Cl)c1)N1CCc2nc(NCC3(O)CC3)sc2C1]

Rt (Method B) 2.46 mins, m/z 397/399 [M+H]+

¹H NMR (400 MHz, DMSO-d6) δ 8.79 (s, 1H), 7.75 (dd, J=6.9, 2.6 Hz, 1H), 7.50 (t, J=5.6 Hz, 1H), 7.42 (ddd, J=9.1, 4.3, 2.7 Hz, 1H), 7.29 (t, J=9.1 Hz, 1H), 5.42 (s, 1H), 4.46-4.41 (m, 2H), 3.75-3.68 (m, 2H), 3.38-3.33 (m, 2H), 2.57-2.51 (m, 2H), 0.58-0.49 (m, 4H). cl Example 13

### N-(4-fluoro-3-methylphenyl)-2-{[(1-hydroxycyclobutyl)methyl]amino}-4H,5H,6H,7H-[1,3]thiazolo[5,4-c]pyridine-5-carboxamide

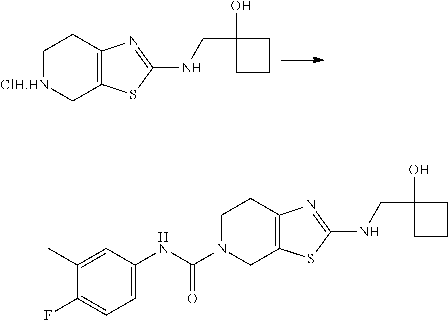

To a solution of CDI (0.059 g, 0.363 mmol) in dichloromethane (1 mL) under Na was added solution of 4-fluoro-3-methyl-aniline (0.045 g, 0.363 mmol) in dichloromethane (1 mL) was added. The mixtures were stirred for 2 h, then a solution of 1-(((4,5,6,7-tetrahydrothiazolo[5,4-c]pyridin-2-yl)amino)methyl)cyclobutan-1-ol hydrochloride (0.100 g, 0.363 mmol) in dichloromethane (2 mL) was added, followed by triethylamine (0.111 mL, 0.798 mmol). Solvents were removed under a stream of nitrogen, and the residue purified by HPLC to give N-(3-methyl-4-fluorophenyl)-2-{[(1-hydroxycyclobutyl)methyl]amino}-4H,5H,6H,7H-[1,3]thiazolo [5,4-c]pyridine-5-carboxamide as a pale yellow solid (59 mg, 42% yield).

Rt (Method B) 2.42 mins, m/z 391 [M+H]+

¹H NMR (400 MHz, DMSO-d6) δ 8.56 (s, 1H), 7.39 (t, J=5.6 Hz, 1H), 7.35 (dd, J=7.1, 2.8 Hz, 1H), 7.29-7.22 (m, 1H), 6.99 (t, J=9.2 Hz, 1H), 5.28 (s, 1H), 4.46-4.38 (m, 2H), 3.74-3.67 (m, 2H), 3.32-3.28 (m, 2H), 2.57-2.51 (m, 2H), 2.21-2.14 (m, 3H), 2.05-1.85 (m, 4H), 1.67-1.56 (m, 1H), 1.53-1.38 (m, 1H).

### Example 14

### N-(3-cyano-4-fluorophenyl)-2-{[(1-hydroxycyclobutyl)methyl]amino}-4H,5H,6H,7H-[1,3]thiazolo[5,4-c]pyridine-5-carboxamide

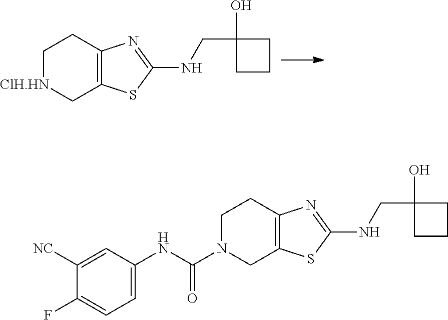

To a solution of CDI (0.059 g, 0.363 mmol) in dichloromethane (1 mL) under Na was added solution of 5-amino-2-fluorobenzonitrile (0.049 g, 0.363 mmol) in dichloromethane (1 mL) was added. The mixtures were stirred for 2h, then a solution of 1-(((4,5,6,7-tetrahydrothiazolo[5,4-c]pyridin-2-yl)amino)methyl)cyclobutan-1-ol hydrochloride (0.100 g, 0.363 mmol) in dichloromethane (2 mL) was added, followed by triethylamine (0.111 mL, 0.798 mmol). Solvents were removed under a stream of nitrogen, and the residue purified by HPLC to give N-(3-cyano-4-fluorophenyl)-2-{[(1-hydroxycyclobutyl)methyl]amino}-4H,5H,6H,7H-[1,3]thiazolo[5,4-c]pyridine-5-carboxamide as a pale yellow solid (50 mg, 34% yield).

Rt (Method B) 2.37 mins, m/z 402 [M+H]+

¹H NMR (400 MHz, DMSO-d6) δ 8.97 (s, 1H), 7.95 (dd, J=5.8, 2.8 Hz, 1H), 7.83-7.75 (m, 1H), 7.47-7.38 (m, 2H), 5.27 (s, 1H), 4.48-4.42 (m, 2H), 3.77-3.71 (m, 2H), 3.34-3.32 (m, 2H), 2.59-2.52 (m, 2H), 2.05-1.86 (m, 4H), 1.67-1.57 (m, 1H), 1.51-1.39 (m, 1H).

### Example 15

### N-(3-chloro-4-fluorophenyl)-2-(4-hydroxycyclohexaneamido)-4H,5H,6H,7H-[1,3]thiazolo[5,4-c]pyridine-5-carboxamide

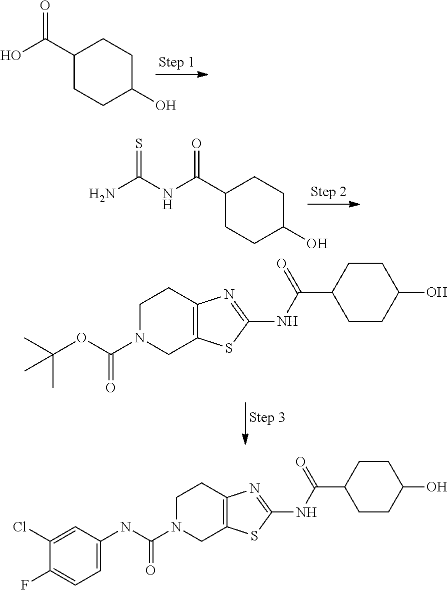

Step 1: To a solution of 4-hydroxycyclohexane-1-carboxylic acid (1 g, 6.94 mmol) in dry N,N-dimethylformamide (10 mL) was added CDI (1.125 g, 6.94 mmol), followed by thiourea (1.056 g, 13.87 mmol). The mixture was stirred at r.t. for 2 hours, then at 50° C. for 3 hours and then 80° C. overnight. NaHCO₃ and EtOAc (50 mL) were added. The layers were separated, the aqueous layer extracted with EtOAc (50 mL). The combined organic extracts were washed with brine (3×50 mL), dried over Na₂SO₄, filtered and concentrated in vacuo to give a wet yellow solid. CH₂Cl₂ was added and the precipitate that formed was removed by filtration. The filtrate was concentrated and purified by flash chromatography (0-10% MeOH in CH₂Cl₂) to give (4-hydroxycyclohexanecarbonyl)thiourea as a white solid (74 mg, 5% yield).

Step 2: A mixture of tert-butyl 3-bromo-4-oxopiperidine-1-carboxylate (120 mg, 0.430 mmol), (4-hydroxycyclohexanecarbonyl)thiourea (87 mg, 0.430 mmol) and sodium bicarbonate (54.2 mg, 0.645 mmol) stirred at 80° C. for 7 hours. The reaction mixture was cooled and then concentrated in vacuo. CH₂Cl₂ was added the solution was filtered. The filtrate was concentrated and purified by flash chromatography (0-10% MeOH in CH2Cl2) to give tert-butyl 2-(4-hydroxycyclohexaneamido)-4H,5H,6H,7H-[1,3]thiazolo[5,4-c]pyridine-5-carboxylate as a colourless foam (85 mg, 46% yield).

Step 3: To tert-butyl 2-(4-hydroxycyclohexane-1-carboxamido)-6,7-dihydrothiazolo[5,4-c]pyridine-5(4H)-carboxylate (85 mg, 0.198 mmol) was added 4M HCl in dioxane (2 mL, 8.00 mmol). The mixture was stirred at r.t. for 2 hours, then concentrated in vacuo and co-evaporated with toluene (twice) and EtOAc. The residue obtained (4-hydroxy-N-(4,5,6,7-tetrahydrothiazolo[5,4-c]pyridin-2-yl)cyclohexane-1-carboxamide hydrochloride) (63 mg, 0.198 mmol) in dry N,N-dimethylformamide (1 mL) were added 2-chloro-1-fluoro-4-isocyanatobenzene (0.025 mL, 0.198 mmol) and TEA (0.04 mL, 0.297 mmol). The mixture was stirred at r.t. overnight. NaHCO₃ and EtOAc (10 mL) were added. The layers were separated, and the aqueous layer was extracted with EtOAc (10 mL). The combined organic extracts were washed with brine (3×10 mL), dried over Na₂SO₄, filtered and concentrated in vacuo. The residue was purified by flash chromatography (10-100% EtOAc in heptane) to give N-(3-chloro-4-fluorophenyl)-2-(4-hydroxycyclohexaneamido)-4H,5H,6H,7H-[1,3]thiazolo[5,4-c]pyridine-5-carboxamide as a white solid (10 mg, 11% yield).

Rt (Method A) 2.94 mins, m/z 453/455 [M+H]+

¹H NMR (400 MHz, DMSO-d6) δ 11.92 (s, 1H), 8.85 (s, 1H), 7.75 (dd, J=6.9, 2.6 Hz, 1H), 7.49-7.37 (m, 1H), 7.30 (t, J=9.1 Hz, 1H), 4.92-4.24 (m, 3H), 3.78 (t, J=5.7 Hz, 2H), 3.49-3.36 (m, 1H), 2.69 (t, J=5.7 Hz, 2H), 2.43-2.29 (m, 1H), 1.96-1.69 (m, 4H), 1.52-1.36 (m, 2H), 1.21-1.06 (m, 2H).

### Example 16

### N-[2-(difluoromethyl)pyridin-4-yl]-2-{[(1r,3r)-3-hydroxycyclobutyl]amino}-4H,5H,6H,7H-[1,3]thiazolo[5,4-c]pyridine-5-carboxamide

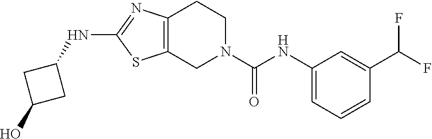 [SMILES: O=C(Nc1cccc(C(F)F)c1)N1CCc2nc(N[C@H]3C[C@H](O)C3)sc2C1]

Rt (Method A2) 2.62 mins, m/z 396 [M+H]+

¹H NMR (400 MHz, DMSO-d6) δ 9.32 (s, 1H), 8.41 (d, J=5.6 Hz, 1H), 7.85 (d, J=2.1 Hz, 1H), 7.76 (d, J=6.3 Hz, 1H), 7.64 (dd, J=5.6, 2.1 Hz, 1H), 6.85 (t, J=55.2 Hz, 1H), 5.04 (d, J=5.5 Hz, 1H), 4.52-4.45 (m, 2H), 4.26 (p, J=6.0 Hz, 1H), 4.00 (h, J=6.1 Hz, 1H), 3.75 (t, J=5.7 Hz, 2H), 2.57 (t, J=5.8 Hz, 2H), 2.14 (t, J=6.1 Hz, 4H).

### Example 17

### N-(3-chloro-4-fluorophenyl)-2-cyclopropanesulfonamido-4H,5H,6H,7H-[1,3]thiazolo[5,4-c]pyridine-5-carboxamide

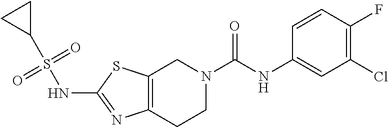 [SMILES: O=C(Nc1ccc(F)c(Cl)c1)N1CCc2nc(NS(=O)(=O)C3CC3)sc2C1]

Rt (Method H) 1.37 mins, m/z 431/433 [M+H]+

¹H NMR (400 MHz, DMSO-d6) δ 12.48 (s, 1H), 8.87 (s, 1H), 7.73 (dd, J=6.9, 2.6 Hz, 1H), 7.48-7.37 (m, 1H), 7.31 (t, J=9.1 Hz, 1H), 4.46-4.34 (m, 2H), 3.75 (t, J=5.6 Hz, 2H), 2.61-2.52 (m, 3H), 0.94-0.82 (m, 4H).

### Example 18

### N-(3-chloro-4-fluorophenyl)-2-(1-methylcyclopropanesulfonamido)-4H,5H,6H,7H-[1,3]thiazolo[5,4-c]pyridine-5-carboxamide

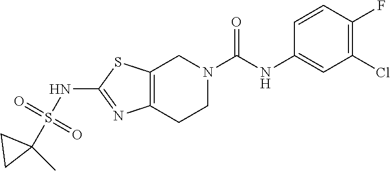 [SMILES: CC1(S(=O)(=O)Nc2nc3c(s2)CN(C(=O)Nc2ccc(F)c(Cl)c2)CC3)CC1]

Rt (Method H) 1.44 mins, m/z 445/447 [M+H]+

¹H NMR (400 MHz, DMSO-d6) δ 12.77-12.15 (m, 1H), 8.86 (s, 1H), 7.73 (dd, J=6.9, 2.6 Hz, 1H), 7.44-7.37 (m, 1H), 7.31 (t, J=9.1 Hz, 1H), 4.42-4.36 (m, 2H), 3.74 (t, J=5.7 Hz, 2H), 2.56-2.52 (m, 2H), 1.38 (s, 3H), 1.19-1.11 (m, 2H), 0.77-0.71 (m, 2H).

### Example 19

### N-[2-(difluoromethyl)pyridin-4-yl]-2-{[(1-hydroxycyclobutyl)methyl]amino}-4H,5H,6H,7H-[1,3]thiazolo[5,4-c]pyridine -5-carboxamide

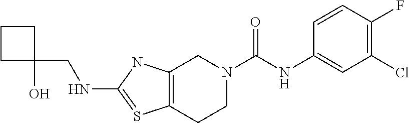

Rt (Method A2) 2.88 mins, m/z 410 [M+H]+

¹H NMR (400 MHz, DMSO-d6) δ 9.32 (s, 1H), 8.41 (d, J=5.6 Hz, 1H), 7.85 (d, J=2.1 Hz, 1H), 7.64 (dd, J=5.8, 2.0 Hz, 1H), 7.43 (t, J=5.6 Hz, 1H), 6.85 (t, J=55.2 Hz, 1H), 5.28 (s, 1H), 4.50-4.45 (m, 2H), 3.75 (t, J=5.7 Hz, 2H), 3.32-3.29 (m, 2H), 2.59-2.52 (m, 2H), 2.04-1.85 (m, 4H), 1.69-1.55 (m, 1H), 1.50-1.40 (m, 1H).

### Example 20

### N-(3-chloro-4-fluorophenyl)-2-[(1r,3r)-3-hydroxycyclobutaneamido]-4H,5H,6H,7H-[1,3]thiazolo[5,4-c]pyridine-5-carboxamide

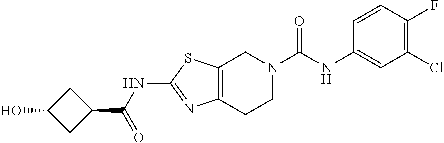 [SMILES: O=C(Nc1ccc(F)c(Cl)c1)N1CCc2nc(NC(=O)[C@H]3C[C@H](O)C3)sc2C1]

Rt (Method H) 1.32 mins, m/z 425/427 [M+H]+

¹H NMR (400 MHz, DMSO-d6) δ 11.87 (s, 1H), 8.85 (s, 1H), 7.74 (dd, J=6.9, 2.6 Hz, 1H), 7.45-7.39 (m, 1H), 7.29 (t, J=9.1 Hz, 1H), 5.15 (d, J=6.2 Hz, 1H), 4.64-4.58 (m, 2H), 4.27 (p, J=6.7 Hz, 1H), 3.77 (t, J=5.8 Hz, 2H), 3.18-3.08 (m, 1H), 2.68 (t, J=5.9 Hz, 2H), 2.40-2.31 (m, 2H), 2.12-2.02 (m, 2H).

### Example 21

### (6S)-N-(3-chloro-4-fluorophenyl)-6-methyl-2-[(1r,3r)-3-hydroxycyclobutaneamido]-4H,5H,6H,7H-[1,3]thiazolo[5,4-c]pyridine-5-carboxamide

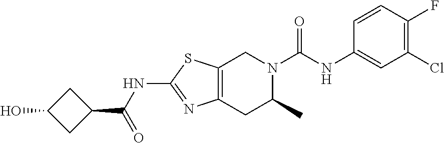 [SMILES: C[C@H]1Cc2nc(NC(=O)[C@H]3C[C@H](O)C3)sc2CN1C(=O)Nc1ccc(F)c(Cl)c1]

Rt (Method H) 1.37 mins, m/z 439/441 [M+H]+

¹H NMR (400 MHz, DMSO-d6) δ 11.87 (s, 1H), 8.80 (s, 1H), 7.75 (dd, J=6.9, 2.6 Hz, 1H), 7.46-7.39 (m, 1H), 7.29 (t, J=9.1 Hz, 1H), 5.16 (d, 1H), 5.02-4.93 (m, 1H), 4.81 (p, J=6.4 Hz, 1H), 4.33-4.23 (m, 1H), 4.23-4.14 (m, 1H), 3.19-3.10 (m, 1H), 2.95-2.85 (m, 1H), 2.43-2.31 (m, 2H), 2.15-2.02 (m, 2H), 1.12 (d, J=6.7 Hz, 3H).

Selected compounds of the invention were assayed in capsid assembly and HBV replication assays, as described below and a representative group of these active compounds is shown in Table 1.

Biochemical Capsid Assembly Assay

The screening for assembly effector activity was done based on a fluorescence quenching assay published by Zlotnick et al. (2007). The C-terminal truncated core protein containing 149 amino acids of the N-terminal assembly domain fused to a unique cysteine residue at position 150 and was expressed in E. coli using the pET expression system (Merck Chemicals, Darmstadt). Purification of core dimer protein was performed using a sequence of size exclusion chromatography steps. In brief, the cell pellet from 1 L BL21 (DE3) Rosetta2 culture expressing the coding sequence of core protein cloned NdeI/XhoI into expression plasmid pET21b was treated for 1 h on ice with a native lysis buffer (Qproteome Bacterial Protein Prep Kit; Qiagen, Hilden). After a centrifugation step the supernatant was precipitated during 2 h stirring on ice with 0.23 g/ml of solid ammonium sulfate. Following further centrifugation the resulting pellet was resolved in buffer A (100 mM Tris, pH 7.5; 100 mM NaCl; 2 mM DTT) and was subsequently loaded onto a buffer A equilibrated CaptoCore 700 column (GE HealthCare, Frankfurt). The column flow through containing the assembled HBV capsid was dialyzed against buffer N (50 mM NaHCO₃ pH 9.6; 5 mM DTT) before urea was added to a final concentration of 3M to dissociate the capsid into core dimers for 1.5 h on ice. The protein solution was then loaded onto a 1 L Sephacryl 5300 column. After elution with buffer N core dimer containing fractions were identified by SDS-PAGE and subsequently pooled and dialyzed against 50 mM HEPES pH 7.5; 5 mM DTT. To improve the assembly capacity of the purified core dimers a second round of assembly and disassembly starting with the addition of 5 M NaCl and including the size exclusion chromatography steps described above was performed. From the last chromatography step core dimer containing fractions were pooled and stored in aliquots at concentrations between 1.5 to 2.0 mg/ml at −80° C.

Immediately before labelling the core protein was reduced by adding freshly prepared DTT in a final concentration of 20 mM. After 40 mM incubation on ice storage buffer and DTT was removed using a Sephadex G-25 column (GE HealthCare, Frankfurt) and 50 mM HEPES, pH 7.5. For labelling 1.6 mg/ml core protein was incubated at 4° C. and darkness overnight with BODIPY-FL maleimide (Invitrogen, Karlsruhe) in a final concentration of 1 mM. After labelling the free dye was removed by an additional desalting step using a Sephadex G-25 column. Labelled core dimers were stored in aliquots at 4° C. In the dimeric state the fluorescence signal of the labelled core protein is high and is quenched during the assembly of the core dimers to high molecular capsid structures. The screening assay was performed in black 384 well microtiter plates in a total assay volume of 10 μl using 50 mM HEPES pH 7.5 and 1.0 to 2.0 μM labelled core protein. Each screening compound was added in 8 different concentrations using a 0.5 log-unit serial dilution starting at a final concentration of 100 μM, 31.6 μM or 10 μM, In any case the DMSO concentration over the entire microtiter plate was 0.5%. The assembly reaction was started by the injection of NaCl to a final concentration of 300 μM which induces the assembly process to approximately 25% of the maximal quenched signal. 6 min after starting the reaction the fluorescence signal was measured using a Clariostar plate reader (BMG Labtech, Ortenberg) with an excitation of 477 nm and an emission of 525 nm. As 100% and 0% assembly control HEPES buffer containing 2.5 M and 0 M NaCl was used. Experiments were performed thrice in triplicates. EC₅₀ values were calculated by non-linear regression analysis using the Graph Pad Prism 6 software (GraphPad Software, La Jolla, USA).

Determination of HBV DNA from the Supernatants of HepAD38 Cells

The anti-HBV activity was analysed in the stable transfected cell line HepAD38, which has been described to secrete high levels of HBV virion particles (Ladner et al., 1997). In brief, HepAD38 cells were cultured at 37° C. at 5% CO₂ and 95% humidity in 200 μl maintenance medium, which was Dulbecco's modified Eagle's medium/Nutrient Mixture F-12 (Gibco, Karlsruhe), 10% fetal bovine serum (PAN Biotech Aidenbach) supplemented with 50 μg/ml penicillin/streptomycin (Gibco, Karlsruhe), 2 mM L-glutamine (PAN Biotech, Aidenbach), 400 μg/ml G418 (AppliChem, Darmstadt) and 0.3 μg/ml tetracycline. Cells were subcultured once a week in a 1:5 ratio, but were usually not passaged more than ten times. For the assay 60,000 cells were seeded in maintenance medium without any tetracycline into each well of a 96-well plate and treated with serial half-log dilutions of test compound. To minimize edge effects the outer 36 wells of the plate were not used but were filled with assay medium. On each assay plate six wells for the virus control (untreated HepAD38 cells) and six wells for the cell control (HepAD38 cells treated with 0.3 μg/ml tetracycline) were allocated, respectively. In addition, one plate set with reference inhibitors like BAY 41-4109, entecavir, and lamivudine instead of screening compounds were prepared in each experiment. In general, experiments were performed thrice in triplicates. At day 6 HBV DNA from 100 μl filtrated cell culture supernatant (AcroPrep Advance 96 Filter Plate, 0.45 μM Supor membran, PALL GmbH, Dreieich) was automatically purified on the MagNa Pure LC instrument using the MagNA Pure 96 DNA and Viral NA Small Volume Kit (Roche Diagnostics, Mannheim) according to the instructions of the manufacturer. EC50 values were calculated from relative copy numbers of HBV DNA In brief, 5 μl of the 100 μl eluate containing HBV DNA were subjected to PCR LC480 Probes Master Kit (Roche) together with 1 μM antisense primer tgcagaggtgaagcgaagtgcaca, 0.5 μM sense primer gacgtcctttgtttacgtcccgtc, 0.3 μM hybprobes acggggcgcacctctctttacgcgg-FL and LC640-ctccccgtctgtgccttctcatctgc-PH (TIBMolBiol, Berlin) to a final volume of 12.5 μl. The PCR was performed on the Light Cycler 480 real time system (Roche Diagnostics, Mannheim) using the following protocol: Pre-incubation for 1 min at 95° C., amplification: 40 cycles×(10 sec at 95° C., 50 sec at 60° C., 1 sec at 70° C.), cooling for 10 sec at 40° C. Viral load was quantitated against known standards using HBV plasmid DNA of pCH-9/3091 (Nassal et al., 1990, Cell 63: 1357-1363) and the LightCycler 480 SW 1.5 software (Roche Diagnostics, Mannheim) and EC₅₀ values were calculated using non-linear regression with GraphPad Prism 6 (GraphPad Software Inc., La Jolla, USA).

Cell Viability Assay

Using the AlamarBlue viability assay cytotoxicity was evaluated in HepAD38 cells in the presence of 0.3 μg/ml tetracycline, which blocks the expression of the HBV genome. Assay condition and plate layout were in analogy to the anti-HBV assay, however other controls were used. On each assay plate six wells containing untreated HepAD38 cells were used as the 100% viability control, and six wells filled with assay medium only were used as 0% viability control. In addition, a geometric concentration series of cycloheximide starting at 60 μM final assay concentration was used as positive control in each experiment. After six days incubation period Alamar Blue Presto cell viability reagent (ThermoFisher, Dreieich) was added in 1/11 dilution to each well of the assay plate. After an incubation for 30 to 45 min at 37° C. the fluorescence signal, which is proportional to the number of living cells, was read using a Tecan Spectrafluor Plus plate reader with an excitation filter 550 nm and emission filter 595 nm, respectively. Data were normalized into percentages of the untreated control (100% viability) and assay medium (0% viability) before CC50 values were calculated using non-linear regression and the GraphPad Prism 6.0 (GraphPad Software, La Jolla, USA). Mean EC₅₀ and CC₅₀ values were used to calculate the selectivity index (SI=CC₅₀/EC₅₀) for each test compound.

TABLE 1
Biochemical and antiviral activities

| Example | CC₅₀ (μM) | Cell Activity | Assembly Activity |
|---|---|---|---|
| Example 1 | >10 | +++ | A |
| Example 2 | >10 | +++ | A |
| Example 3 | >10 | +++ | A |
| Example 4 | >10 | +++ | A |
| Example 5 | >10 | +++ | A |
| Example 6 | >10 | +++ | A |
| Example 7 | >10 | +++ | A |
| Example 8 | >10 | +++ | A |
| Example 9 | >10 | +++ | A |
| Example 10 | >10 | +++ | A |
| Example 11 | >10 | +++ | A |
| Example 12 | >10 | +++ | A |
| Example 13 | >10 | +++ | A |
| Example 14 | >10 | +++ | A |
| Example 15 | >10 | +++ | A |
| Example 16 | >10 | ++ | C |
| Example 17 | >10 | ++ | C |
| Example 18 | >10 | ++ | C |
| Example 19 | >10 | ++ | C |
| Example 20 | >10 | +++ | A |
| Example 21 | >10 | +++ | A |

In Table 1, “+++” represents an EC₅₀ <1 μM; “++” represents 1 μM < EC₅₀ < 10 μM; “+” represents EC₅₀ <100 μM (Cell activity assay)

In Vivo Efficacy Models

HBV research and preclinical testing of antiviral agents are limited by the narrow species- and tissue-tropism of the virus, the paucity of infection models available and the restrictions imposed by the use of chimpanzees, the only animals fully susceptible to HBV infection. Alternative animal models are based on the use of HBV-related hepadnaviruses and various antiviral compounds have been tested in woodchuck hepatitis virus (WHV) infected woodchucks or in duck hepatitis B virus (DHBV) infected ducks or in woolly monkey HBV (WM-HBV) infected tupaia (overview in Dandri et al., 2017, Best Pract Res Clin Gastroenterol 31, 273-279). However, the use of surrogate viruses has several limitations. For example is the sequence homology between the most distantly related DHBV and HBV is only about 40% and that is why core protein assembly modifiers of the HAP family appeared inactive on DHBV and WHV but efficiently suppressed HBV (Campagna et al., 2013, J. Virol. 87, 6931-6942). Mice are not HBV permissive but major efforts have focused on the development of mouse models of HBV replication and infection, such as the generation of mice transgenic for the human HBV (HBV tg mice), the hydrodynamic injection (HDI) of HBV genomes in mice or the generation of mice having humanized livers and/or humanized immune systems and the intravenous injection of viral vectors based on adenoviruses containing HBV genomes (Ad-HBV) or the adenoassociated virus (AAV-HBV) into immune competent mice (overview in Dandri et al., 2017, Best Pract Res Clin Gastroenterol 31, 273-279). Using mice transgenic for the full HBV genome the ability of murine hepatocytes to produce infectious HBV virions could be demonstrated (Guidotti et al., 1995, J. Virol., 69: 6158-6169). Since transgenic mice are immunological tolerant to viral proteins and no liver injury was observed in HBV-producing mice, these studies demonstrated that HBV itself is not cytopathic. HBV transgenic mice have been employed to test the efficacy of several anti-HBV agents like the polymerase inhibitors and core protein assembly modifiers (Weber et al., 2002, Antiviral Research 54 69-78; Julander et al., 2003, Antivir. Res., 59: 155-161), thus proving that HBV transgenic mice are well suitable for many type of preclinical antiviral testing in vivo.

As described in Paulsen et al., 2015, PLOSone, 10: e0144383 HBV-transgenic mice (Tg [HBV1.3 fsX⁻3′5′]) carrying a frameshift mutation (GC) at position 2916/2917 could be used to demonstrate antiviral activity of core protein assembly modifiers in vivo. In brief, The HBV-transgenic mice were checked for HBV-specific DNA in the serum by qPCR prior to the experiments (see section “Determination of HBV DNA from the supernatants of HepAD38 cells”). Each treatment group consisted of five male and five female animals approximately 10 weeks age with a titer of 10⁷-10⁸ virions per ml serum. Compounds were formulated as a suspension in a suitable vehicle such as 2% DMSO/98% tylose (0.5% Methylcellulose/99.5% PBS) or 50% PEG400 and administered per os to the animals one to three times/day for a 10 day period. The vehicle served as negative control, whereas 1 μg/kg entecavir in a suitable vehicle was the positive control. Blood was obtained by retro bulbar blood sampling using an Isoflurane Vaporizer. For collection of terminal heart puncture six hours after the last treatment blood or organs, mice were anaesthetized with isoflurane and subsequently sacrificed by CO₂ exposure. Retro bulbar (100-150 μl) and heart puncture (400-500 μl) blood samples were collected into a Microvette 300 LH or Microvette 500 LH, respectively, followed by separation of plasma via centrifugation (10 min, 2000 g, 4° C.). Liver tissue was taken and snap frozen in liquid N₂. All samples were stored at −80° C. until further use. Viral DNA was extracted from 50 μl plasma or 25 mg liver tissue and eluted in 50 μl AE buffer (plasma) using the DNeasy 96 Blood & Tissue Kit (Qiagen, Hilden) or 320 pl AE buffer (liver tissue) using the DNeasy Tissue Kit (Qiagen, Hilden) according to the manufacturer's instructions. Eluted viral DNA was subjected to qPCR using the LightCycler 480 Probes Master PCR kit (Roche, Mannheim) according to the manufacturer's instructions to determine the HBV copy number. HBV specific primers used included the forward primer 5′-CTG TAC CAA ACC TTC GGA CGG-3′, the reverse primer 5′-AGG AGA AAC GGG CTG AGG C-3′ and the FAM labelled probe FAM-CCA TCA TCC TGG GCT TTC GGA AAA TT-BBQ. One PCR reaction sample with a total volume of 20 μl contained 5 μl DNA eluate and 15 μl master mix (comprising 0.3 μM of the forward primer, 0.3 μM of the reverse primer, 0.15 μM of the FAM labelled probe). qPCR was carried out on the Roche LightCycler1480 using the following protocol: Pre-incubation for 1 min at 95° C., amplification: (10 sec at 95° C., 50 sec at 60° C., 1 sec at 70° C.)×45 cycles, cooling for 10 sec at 40° C. Standard curves were generated as described above. All samples were tested in duplicate. The detection limit of the assay is 50 HBV DNA copies (using standards ranging from 250-2.5×107 copy numbers). Results are expressed as HBV DNA copies/10 μl plasma or HBV DNA copies/100 ng total liver DNA (normalized to negative control).

It has been shown in multiple studies that not only transgenic mice are a suitable model to proof the antiviral activity of new chemical entities in vivo the use of hydrodynamic injection of HBV genomes in mice as well as the use of immune deficient human liver chimeric mice infected with HBV positive patient serum have also frequently used to profile drugs targeting HBV (Li et al., 2016, Hepat. Mon. 16: e34420; Qiu et al., 2016, J. Med. Chem. 59: 7651-7666; Lutgehetmann et al., 2011, Gastroenterology, 140: 2074-2083). In addition chronic HBV infection has also been successfully established in immunecompetent mice by inoculating low doses of adenovirus- (Huang et al., 2012, Gastroenterology 142: 1447-1450) or adeno-associated virus (AAV) vectors containing the HBV genome (Dion et al., 2013, J Virol. 87: 5554-5563). This models could also be used to demonstrate the in vivo antiviral activity of novel anti-HBV agents.

1. A compound of Formula I 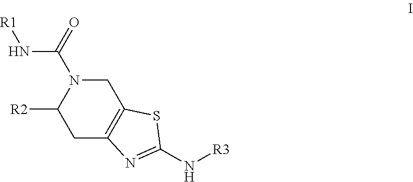
in which
R1 is phenyl or pyridyl, optionally substituted once, twice, or thrice by halo, C1-C4-alkyl, C3-C6-cycloalkyl, C1-C4-haloalkyl or C≡N
R2 is H or methyl
R3 is selected from the group comprising H, D, C1-C6-alkyl, C3-C6-cycloalkyl, C3-C7-heterocycloalkyl, C2-C6-aminoalkyl, SO₂—C1-C6-alkyl, SO₂—C3-C7-cycloalkyl, SO₂—C3-C7-heterocycloalkyl, SO₂—C2-C6-hydroxyalkyl, SO₂—C2-C6-alkyl-O—C1-C6-alkyl, SO₂—C1-C4-carboxyalkyl, SO₂-aryl, SO₂-heteroaryl, SO₂—N(R12)(R13), C(═O)R4, C(═O)N(R12)(R13), C(═O)C(═O)N(R12)(R13), and C2-C6-hydroxyalkyl, optionally substituted with 1, 2, or 3 groups each independently selected from OH, halo, NH₂, acyl, SO₂CH₃, carboxy, carboxyl ester, carbamoyl, substituted carbamoyl, C6-aryl, heteroaryl, C1-C6-alkyl, C3-C6-cycloalkyl, C3-C7-heterocycloalkyl, C1-C6-haloalkyl, C1-C6-alkoxy, C1-C6-alkyl-O—C1-C6-alkyl, C1-C6-hydroxyalkyl, and C2-C6 alkenyloxy, preferably C1-C6-alkyl, C3-C6-cycloalkyl, C3-C7-heterocycloalkyl and C2-C6-hydroxyalkyl
R4 is selected from the group comprising C1-C6-alkyl, C1-C6-hydroxyalkyl, C1-C6-alkyl-O—C1-C6-alkyl, C3-C7-cycloalkyl, C1-C4-carboxyalkyl, C3-C7-heterocycloalkyl, C6-aryl, and heteroaryl optionally substituted with 1, 2, or 3 groups each independently selected from OH, halo, NH₂, acyl, SO₂CH₃, SO₃H, carboxy, carboxyl ester, carbamoyl, substituted carbamoyl, C6-aryl, heteroaryl, C1-C6-alkyl, C3-C6-cyclo alkyl, C3-C7-heterocyclo alkyl, C1-C6-haloalkyl, C1-C6-alkoxy, C1-C6-hydroxyalkyl, and C2-C6 alkenyloxy
R12 and R13 are independently selected from the group comprising H, C1-C6-alkyl, C2-C6-hydroxyalkyl, C2-C6-alkyl-O—C1-C6-alkyl, C3-C7-cycloalkyl, C1-C4-carboxyalkyl, C3-C7-heterocycloalkyl, C6-aryl, and heteroaryl optionally substituted with 1, 2, or 3 groups each independently selected from OH, halo, NH₂, acyl, SO₂CH₃, SO₃H, carboxy, carboxyl ester, carbamoyl, substituted carbamoyl, C6-aryl, heteroaryl, C1-C6-alkyl, C3-C6-cycloalkyl, C3-C7-heterocycloalkyl, C1-C6-haloalkyl, C1-C6-alkoxy, C1-C6-hydroxyalkyl, and C2-C6 alkenyloxy
R12 and R13 are optionally connected to form a C3-C7-heterocycloalkyl ring containing 1 or 2 nitrogen, sulfur or oxygen atoms
or a pharmaceutically acceptable salt thereof or a solvate or a hydrate of a compound of Formula I or the pharmaceutically acceptable salt thereof or a prodrug of a compound of Formula I or a pharmaceutically acceptable salt or a solvate or a hydrate thereof for use in the prevention or treatment of an HBV infection in a subject.

2. A compound of Formula I for use in the prevention or treatment of an HBV infection in a subject according to claim 1, wherein SO₂-aryl is SO₂—C6-aryl, and/or SO₂-heteroaryl is SO₂—C1-C9-heteroaryl and/or heteroaryl is C1-C9-heteroaryl and wherein heteroaryl, SO₂heteroaryl, SO₂-heterocycloalkyl and heterocycloalkyl each has in the ring system 1 to 4 heteroatoms each independently selected from N, O and S,
or a pharmaceutically acceptable salt thereof or a solvate or a hydrate of a compound of Formula I or the pharmaceutically acceptable salt thereof or a prodrug of a compound of Formula I or a pharmaceutically acceptable salt or a solvate or a hydrate thereof.

3. A compound of Formula I for use in the prevention or treatment of an HBV infection in a subject according to claim 1,
or a pharmaceutically acceptable salt thereof or a solvate or a hydrate of a compound of Formula I or the pharmaceutically acceptable salt thereof or a prodrug of a compound of Formula I or a pharmaceutically acceptable salt or a solvate or a hydrate thereof,
wherein the prodrug is selected from the group comprising esters, carbonates, acetyloxy derivatives, amino acid derivatives and phosphoramidate derivatives.

4. A compound of Formula I that is a compound of Formula II for use in the prevention or treatment of an HBV infection in a subject according to claim 1 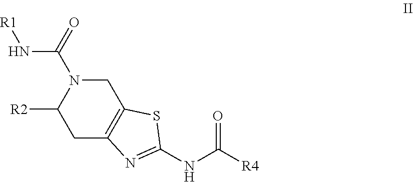
in which
R1 is phenyl or pyridyl, optionally substituted once, twice, or thrice by halo, C1-C4-alkyl, C3-C6-cycloalkyl, C1-C4-haloalkyl or C≡N
R2 is H or methyl
R4 is selected from the group comprising C1-C6-alkyl, C1-C6-hydroxyalkyl, C1-C6-alkyl-O—C1-C6-alkyl, C3-C7-cycloalkyl, C1-C4-carboxyalkyl, C3-C7-heterocycloalkyl, C6-aryl, and heteroaryl optionally substituted with 1, 2, or 3 groups each independently selected from OH, halo, NH₂, acyl, SO₂CH₃, SO₃H, carboxy, carboxyl ester, carbamoyl, substituted carbamoyl, C6-aryl, heteroaryl, C1-C6-alkyl, C3-C6-cycloalkyl, C3-C7-heterocycloalkyl, C1-C6-haloalkyl, C1-C6-alkoxy, C2-C6-hydroxyalkyl, and C2-C6 alkenyloxy
or a pharmaceutically acceptable salt thereof or a solvate or a hydrate of a compound of Formula II or the pharmaceutically acceptable salt thereof or a prodrug of a compound of Formula II or a pharmaceutically acceptable salt or a solvate or a hydrate thereof.

5. A compound of Formula I that is a compound of Formula DI for use in the prevention or treatment of an HBV infection in a subject according to claim 1 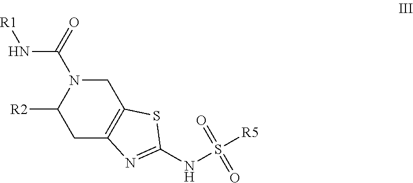
in which
R1 is phenyl or pyridyl, optionally substituted once, twice, or thrice by halo, C1-C4-alkyl, C3-C6-cycloalkyl, C1-C4-haloalkyl or C≡N
R2 is H or methyl
R5 is selected from the group comprising C1-C6-alkyl, C2-C6-hydroxyalkyl, C2-C6-alkyl-O—C1-C6-alkyl, C3-C7-cycloalkyl, C1-C4-carboxyalkyl, C3-C7-heterocycloalkyl, C6-aryl, and heteroaryl, optionally substituted with 1, 2, or 3 groups each independently selected from OH, halo, NH₂, acyl, SO₂CH₃, SO₃H, carboxy, carboxyl ester, carbamoyl, substituted carbamoyl, C6-aryl, heteroaryl, C1-C6-alkyl, C3-C6-cycloalkyl, C3-C7-heterocycloalkyl, C1-C6-haloalkyl, C1-C6-alkoxy, C1-C6-hydroxyalkyl, and C2-C6 alkenyloxy
or a pharmaceutically acceptable salt thereof or a solvate or a hydrate of a compound of Formula III or the pharmaceutically acceptable salt thereof or a prodrug of a compound of Formula III or a pharmaceutically acceptable salt or a solvate or a hydrate thereof.

6. A compound of Formula I that is a compound of Formula IV for use in the prevention or treatment of an HBV infection in a subject according to claim 1 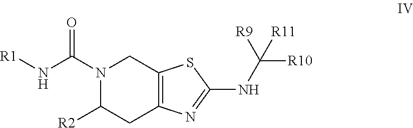
in which
R1 is phenyl or pyridyl, optionally substituted once, twice, or thrice by halo, C1-C4-alkyl, C3-C6-cycloalkyl, C1-C4-haloalkyl or C≡N
R2 is H or methyl
R9, R10 and R11 are independently selected from the group comprising H, C1-C5-hydroxyalkyl, C1-C5-alkyl-O—C1-C6-alkyl, C1-C5-alkyl, C3-C7-cycloalkyl, C1-C3-carboxyalkyl, C3-C7-heterocycloalkyl, C6-aryl, and heteroaryl, wherein C1-C5-alkyl, C1-C5-hydroxyalkyl, C1-C5-alkyl-O—C1-C6-alkyl and C1-C3-carboxyalkyl are optionally substituted with 1, 2, or 3 groups each independently selected from OH, halo, NH₂, acyl, SO₂CH₃, SO₃H, carboxy, carboxyl ester, carbamoyl, substituted carbamoyl, C6-aryl, heteroaryl, C1-C6-alkyl, C3-C6-cycloalkyl, C3-C7-heterocycloalkyl, C1-C6-haloalkyl, C1-C6-alkoxy, C1-C6-hydroxyalkyl, and C2-C6 alkenyloxy
R9 and R10 are optionally connected to form a C3-C7 cycloalkyl ring, or a C4-C7-heterocycloalkyl ring containing 1 or 2 nitrogen, sulfur or oxygen atoms
or a pharmaceutically acceptable salt thereof or a solvate or a hydrate of a compound of Formula IV or the pharmaceutically acceptable salt thereof or a prodrug of a compound of Formula IV or a pharmaceutically acceptable salt or a solvate or a hydrate thereof.

7. A pharmaceutical composition comprising a compound according to claim 1 or a pharmaceutically acceptable salt thereof or a solvate or a hydrate of said compound or the pharmaceutically acceptable salt thereof or a prodrug of said compound or a pharmaceutically acceptable salt or a solvate or a hydrate thereof, together with a pharmaceutically acceptable carrier for use in the prevention or treatment of an HBV infection in a subject.

8. A method of treating an HBV infection in an individual in need thereof, comprising administering to the individual a therapeutically effective amount of a compound according to claim 1 or a pharmaceutically acceptable salt thereof or a solvate or a hydrate of said compound or the pharmaceutically acceptable salt thereof or a prodrug of said compound or a pharmaceutically acceptable salt or a solvate or a hydrate thereof.


SEQUENCE LISTING (7 sequences)

SEQ ID NO: 1 - DNA, 21 bp, Hepatitis B virus
ctgtaccaaaccttcggacgg

SEQ ID NO: 2 - DNA, 18 bp, Hepatitis B virus
aggagaaacgggctgagg

SEQ ID NO: 3 - DNA, 26 bp, Hepatitis B virus
ccatcatcctgggctttcggaaaatt

SEQ ID NO: 4 - DNA, 24 bp, Hepatitis B virus
tgcagaggtgaagcgaagtgcaca

SEQ ID NO: 5 - DNA, 24 bp, Hepatitis B virus
gacgtcctttgtttacgtcccgtc

SEQ ID NO: 6 - DNA, 25 bp, Hepatitis B virus
acggggcgcacctctctttacgcgg

SEQ ID NO: 7 - DNA, 26 bp, Hepatitis B virus
ctccccgtctgtgccttctcatctgc